(19) United States
(12) Patent Application Publication
(10) Pub. No.: US 2024/0067721 A1
(43) Pub. Date: Feb. 29, 2024

(54) NANOBODY TO GLYCOPROTEIN VI

(71) Applicant: The University of Birmingham, (US)

(72) Inventors: Alexandre Slater, Birmingham (GB); Natalie Sarah Poulter, Birmingham (GB); Mark Robert Thomas, Birmingham (GB); Steve Paul Watson, Birmingham (GB)

(21) Appl. No.: 18/269,308

(22) PCT Filed: Dec. 21, 2021
(86) PCT No.: PCT/EP2021/087129

§ 371 Date: Jun. 23, 2023

(30) Foreign Application Priority Data
Dec. 24, 2020 (GB) 2020602.5

(51) Int. Cl. C07K 16/28; A61P 7/02

(52) U.S. Cl. CPC C07K 16/2803; A61P 7/02; C07K 2317/34; C07K 2317/569; C07K 2317/76; C07K 2317/92

(57) ABSTRACT

The present disclosure relates to nanobodies, such as defined by SEQ ID NOS: 1-54, which may be encoded by SEQ ID NOS: 55-110, and which are capable of specifically binding glycoprotein VI (GPVI), as well as uses thereof, which include therapeutic and/or imaging uses.

### FIELD

The present disclosure relates to nanobodies raised against glycoprotein VI (GPVI), as well as uses thereof.

### BACKGROUND

The platelet glycoprotein VI (GPVI) has been identified as an attractive anti-thrombotic target {Andrews, 2014}. GPVI is the major platelet signalling receptor for collagen {Nieswandt, 2003}, and is a receptor for other ligands, including fibrin {Mammadova-Bach, 2015}. GPVI consists of two immunoglobulin (Ig)-like domains (D1 and D2), a highly O-glycosylated and sialylated stalk region, a single spanning trans-membrane helix and a short intracellular tail {Moroi, 2004}. There is a single N-glycosylation site in the N-terminal D1 domain. GPVI signalling requires the FcR γ-chain homodimer, which associates through a salt bridge in the trans-membrane region of GPVI {Zheng, 2001}. Ligand binding occurs through the D1 domain {Lecut, 2004} and leads to phosphorylation of the two conserved tyrosines found within the immunoreceptor tyrosine based activation motif (ITAM) present on the FcR γ-chain by Src family kinases {Ezumi, 1998}. Phosphorylation of the ITAM allows the recruitment of the tyrosine kinase Syk via its tandem SH2 domains and further downstream signalling {Watson, 2005}.

GPVI has been proposed to exist as a monomer and dimer in the membrane {Jung, 2012}. The group of Moroi and Jung reported that recombinant dimeric GPVI, where the extracellular domains are fused to the dimeric Fc domain from IgG, but not monomeric GPVI, binds to collagen with micromolar affinity {Miura, 2002}. They proposed that the differential binding was due either to increased avidity or to the formation of a dimer-specific epitope. The latter was supported by the discovery of dimer-specific antibodies that detected increased expression upon platelet activation {Jung, 2012}. Since GPVI is not present in intracellular stores in platelets, this suggests that the increase in binding is due to a conformational change as a result of dimerisation. The crystal structure of the recombinant GPVI extracellular domain has been solved for both unbound (PDB: 2GI7 and 5OU7) and collagen-related-peptide (CRP) bound forms (PDB: 5OU8 and 5OU9), with both structures revealing a back-to-back dimerisation interface present within the D2 domain. Additionally, collagen is able to cluster GPVI receptors on the platelet membrane surface {Poulter, 2017} and therefore generate higher order oligomers. GPVI-signalling occurs through dimerisation and is increased by further clustering.

The concept of a dimer-specific epitope in GPVI however is not supported by the crystal structure of CRP-bound to GPVI which shows binding in the D1 domain and suggests a 1:1 stoichiometry rather than de novo formation of a binding epitope. Additionally, the site of binding of the dimer-specific antibodies and the mechanism whereby platelet activation leads to an increase in dimerisation are required to establish a full understanding of the role of dimerisation in platelet activation by GPVI.

To-date, two GPVI-blocking agents with distinct mechanisms of action have undergone early phase clinical trials. Revacept is an Fc dimer of GPVI which competes with platelet GPVI for binding to collagen and its other ligands at sites of injury. Revacept has undergone phase I safety, and two phase II trials in patients with a symptomatic carotid artery stenosis (NCT 01645306) or undergoing percutaneous coronary intervention (NCT03312855). The results of the two phase II trials have not yet been reported. ACT017 is a blocking humanised Fab that has undergone phase I safety trial and is currently undergoing a phase II trial in patients with acute ischaemic stroke (NCT03803007). Both treatments are given in addition to current standard of care.

### SUMMARY

The present disclosure is based in part on the development of novel nanobodies raised against GPVI.

Thus, in a first aspect, there is provided a nanobody, or an antigen binding fragment thereof, which is capable of specifically binding GPVI.

The nanobodies of the present disclosure may bind to GPVI with a Kd value (as determined by surface plasmson resonance followed by kinetic analysis) of less than 500 nM, such as less than 250 nM, less than 100 nM, or even less than 50 nM, 25 nM such as less than 10 nM, 5 nM or 1 nM.

The nanobodies of the present disclosure may bind to a region of the GPVI molecule, which is different to the region to which collagen or CRP binds. The residues to which CRP binds are residues R38, E40, R67, Q71, W76 as determined from the crystal structures PDB: 5OU8 and 5OU9. Thus, in one teaching, the nanobodies of the present disclosure do not bind to one or more (such as 2, 3, 4, or 5) of the following GPVI residues: R38, E40, R67, Q71, W76.

The nanobodies of the present disclosure may bind or be in close proximity (for example, one residue either side of the identified residues) to one or more or more of the following residues in human GPVI: E21, S45, R46, Y47, Q48, P56, A57 and S61 (numbering according to uniprot entry Q9HCN6 minus the first 20 amino acids which comprised the signal peptide) (identified binding residues from co-crystallising GPVI with nanobody 2), or may bind or be in close proximity to (for example, one residue either side of the identified residues) at least one or more of the residues S45, R46, Y47, Q48 and/or S61 (numbering according to uniprot Q9HCN6 minus the signal peptide (residues 1-20), or corresponding residues identified in homologous GPVI sequences from other species, which can easily be discerned by the skilled addressee through sequence alignment studies (such as using Clustal Omega). The nanobodies of the present disclosure may bind or be in close proximity to 2, 3, 4, 5, 6, 7 or 8 of the identified residues.

The nanobodies or antigen binding fragment of the present disclosure may comprise at least 1, 2, 3, 4, 5, 6 or more of the following residues, or a conservative substitution in a CDR3 region of the nanobody or antigen binding fragment thereof: S99, P100, Y102, T104, N105, E111, D112, D114, Y115 (numbering in accordance with Nb2 as identified herein). Through use of sequence alignment software, such as Clustal Omega, the skilled addressee can easily discern whether or not a nanobody has an identical residue or conservative substitution at a position which corresponds with the above-identified residues of Nb2.

In one teaching, the nanobody, or an antigen binding fragment thereof, comprises a sequence which is at least 85%, 90%, 95%, 98%, 99%, or 100% identical with SEQ ID NOS: 1-54.

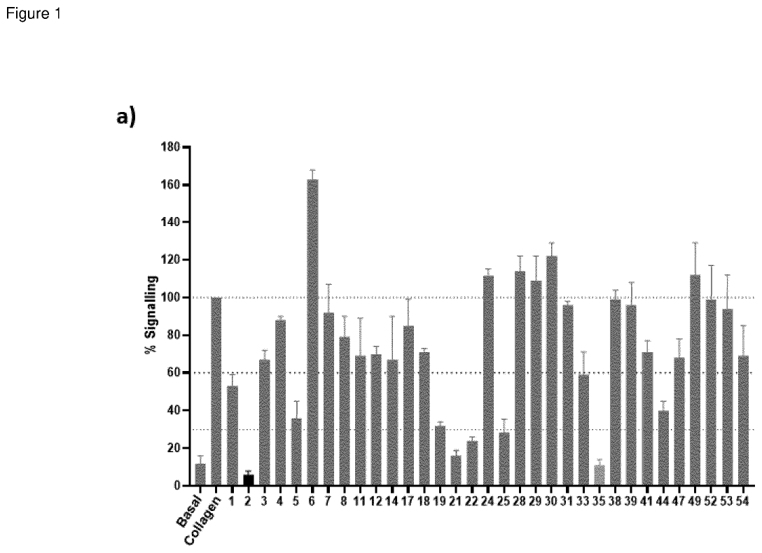

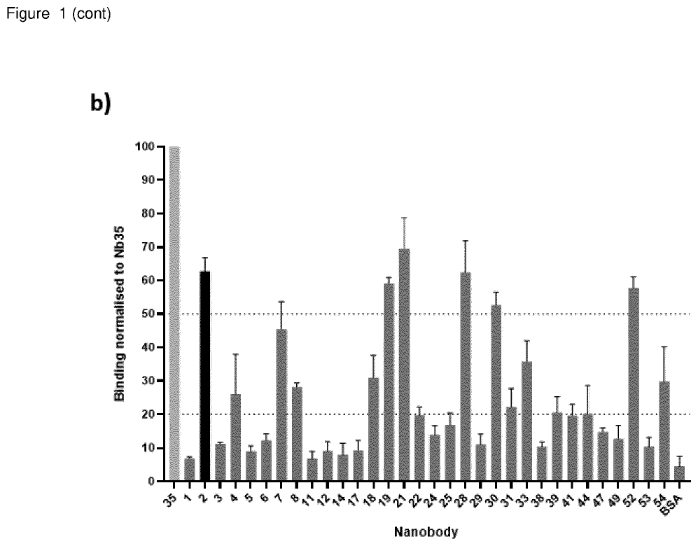

In one teaching, the nanobody, or an antigen binding fragment thereof, comprises a sequence which is at least 85%, 90%, 95%, 98%, 99%, or 100% identical with SEQ ID NOS: 1, 2, 5, 19, , 21, 22, 25, 33, 35, 44. The criteria for this selection was nanobodies that resulted in at least 40% NFAT signal inhibition in a GPVI-transfected cell line assay (FIG. 1a).

In one teaching, the nanobody, or an antigen binding fragment thereof, comprises a sequence which is at least 85%, 90%, 95%, 98%, 99%, or 100% identical with SEQ ID NOS: 2, 21, 22, 25, 35. The criteria for this selection was nanobodies with at least 60% NFAT signal inhibition in a GPVI-transfected cell line assay (FIG. 1a).

In one teaching, the nanobody, or an antigen binding fragment thereof, comprises a sequence which is at least 85%, 90%, 95%, 98%, 99%, or 100% identical with SEQ ID NOS: 2, 21 and 35. The criteria for this selection was nanobodies with at least 80% NFAT signal inhibition in a GPVI-transfected cell line assay (FIG. 1a) and also scored in the top 5 nanobodies binding to GPVI-Fc in an ELISA assay (FIG. 1b).

In one teaching, the nanobody, or an antigen binding fragment thereof, comprises at least the CDR3 sequence, optionally further comprising the CDR1 and/or CDR3 sequences highlighted in the table below.

TABLE 1
Sequence alignment of the top 10 inhibitory nanobodies with CDR
1, 2 and 3 sequences highlighted/underlined in order.

| | |
|---|---|
| CDR1 | CDR2 |

| Nb35 | 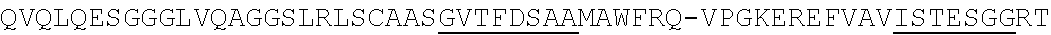 | | 59 |
|---|---|---|---|
| Nb1 | 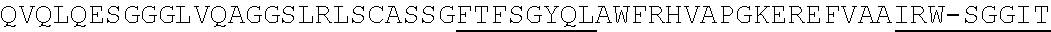 | | 59 |
| Nb21 | 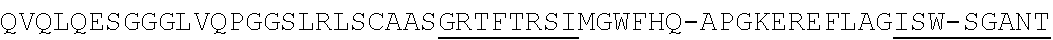 | | 58 |
| Nb22 | 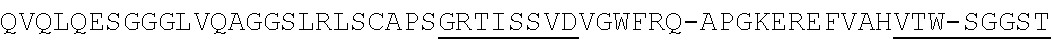 | | 58 |
| Nb33 | 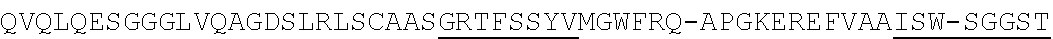 | | 58 |
| Nb5 | 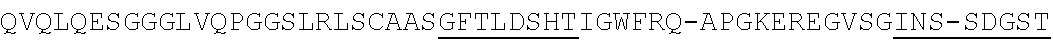 | | 58 |
| Nb44 | 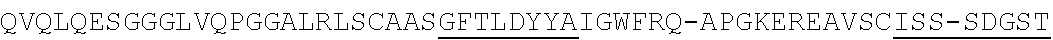 | | 58 |
| Nb2 | 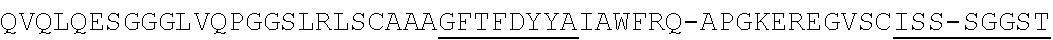 | | 58 |
| Nb25 | 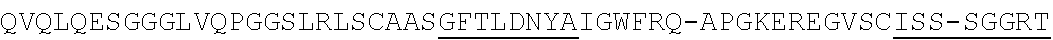 | | 58 |
| NB19 | 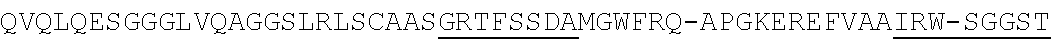 | | 58 |
| | ************* *.:****** :* *: :.**:: .****** :: : *.. * | | |
| | | CDR3 | |

| | | |
|---|---|---|
| Nb35 | 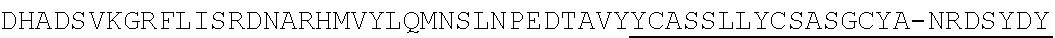 | 118 |
| Nb1 | 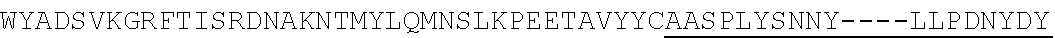 | 115 |
| Nb21 | 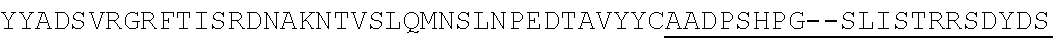 | 116 |
| Nb22 | 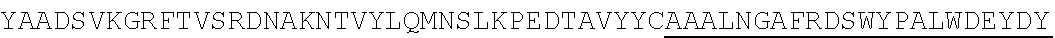 | 118 |
| Nb33 | 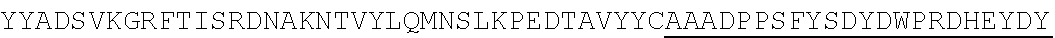 | 118 |
| Nb5 | 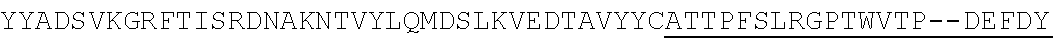 | 116 |
| Nb44 | 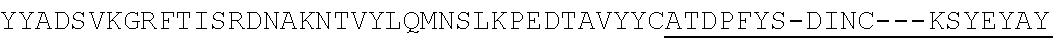 | 114 |
| Nb2 | 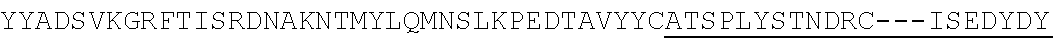 | 115 |
| Nb25 | 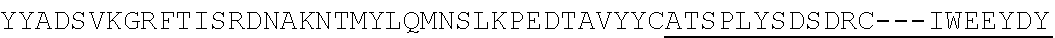 | 115 |
| NB19 | 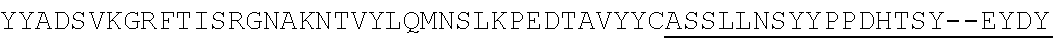 | 116 |
| | ****:*** :::***:: : ***:**: *:*******: .: | |

| | | | |
|---|---|---|---|
| Nb35 | -WGQGTQVTVSSAAAYPYDVPDYGSHHHHHH | 148 | (SEQ ID NO: 35) |
| Nb1 | -WGQGTQVTVSSAAAYPYDVPDYGSHHHHHH | 145 | (SEQ ID NO: 1) |
| Nb21 | -WGRGTQVTVSSAAAYPYDVPDYGSHHHHHH | 146 | (SEQ ID NO: 21) |
| Nb22 | -WGQGTQVTVSSAAAYPYDVPDYGSHHHHHH | 148 | (SEQ ID NO: 22) |
| Nb33 | -WGQGTQVTVSSAAAYPYDVPDYGSHHHHHH | 148 | (SEQ ID NO: 33) |
| Nb5 | -WGQGTQVTVSSAAAYPYDVPDYGSHHHHHH | 146 | (SEQ ID NO: 5) |
| Nb44 | -WGQGTQVTVSSAAAYPYDVPDYGSHHHHHH | 144 | (SEQ ID NO: 44) |
| Nb2 | -WGQGTQVTVSSAAAYPYDVPDYGSHHHHHH | 145 | (SEQ ID NO: 2) |
| Nb25 | -WGQGTQVTVSSAAAYPYDVPDYGSHHHHHH | 145 | (SEQ ID NO: 25) |
| NB19 | -WGQGTQVTVSSAAAYPYDVPDYGSHHHHHH | 146 | (SEQ ID NO: 19) |
| | ***:*************************** | | |

The percent identity between two amino acid sequences can be determined using the algorithm of Myers and Miller {Meyers and Miller 1998} which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch {Needleman and Wunsch 1970} algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

It is to be appreciated the reference to sequence identity refers to the recognisable nanobody sequence and does not therefore include any additional sequences which may be added to the N or C termini of the nanobody, such as proteins or peptides which may be fused with the nanobody sequence.

In another teaching, this disclosure provides nanobodies that bind to the same epitope on GPVI as any of the nanobodies as described herein (i.e., antibodies that have the ability to cross-compete for binding to human GPVI with any of the nanobodies of the disclosure). In certain teachings, the reference nanobody for cross-competition studies can be the Nb2, Nb21 and/or Nb35 nanobodies described herein. Such cross-competing nanobodies can be identified based on their ability to cross-compete with the nanobodies identified herein in standard GPVI binding assays. For example, standard ELISA assays can be used in which a recombinant human GPVI protein is immobilized on a substrate, one of the nanobodies is fluorescently labelled and the ability of non-labelled nanobodies to compete off the binding of the labelled nanobody is evaluated. Additionally, or alternatively, BlAcore analysis can be used to assess the ability of the nanobodies to cross-compete.

Nanobodies are currently the smallest antibody-related molecules with a molecular weight of about 1/10 of a normal antibody. In addition to the antigenic reactivity of monoclonal antibodies, nanobodies possess some unique functional properties, including one or more of the following attributes: small molecular mass; strong stability; good solubility; easy expression; weak immunogenicity; strong penetrability; strong targeting; simple to humanize; and cheap to manufacture as compared to larger antibodies and fragments.

As used herein, the terms “nanobodies” and “single domain antibody (VHH)” have the same meaning referring to antibody fragment consisting of a single monomeric variable antibody domain. Typically, a nanobody consists of only one heavy chain variable region. The term nanobody as used herein refers to monomeric, as well as homo, or hetero-multimeric (e.g. homo or hetero-dimers and tetramers thereof) forms thereof. Mutimeric forms may be generated by including a linker, for example, between individual nanobody domains. An example of a suitable linker is GGGGS, which itself may be as a monomer or multimeric form, such as a trimer. Such nanobodies may find use as antagonists to block GPVI, respectively, with a Kd value of less than 500 nM, 250 nM, 100 nM, 50 nM, 25 nM,10 nM, 5 nM, or 1 nM.

As used herein, the term “variable” means that certain portions of the variable region in a nanobody vary in sequence, from one nanobody to another, which forms the binding and specificity of various specific nanobodies to their particular antigen. However, variability is generally not uniformly distributed throughout the nanobody variable region. It is often concentrated in three segments called complementarity-determining regions (CDRs) or hypervariable regions in the variable regions of the light and heavy chain. The more conserved part of the variable region is called the framework region (FR). The variable regions of the natural heavy and light chains each contain four FR regions, which are substantially in a β-folded configuration, joined by three CDRs which form a linking loop, and in some cases can form a partially β-folded structure. The CDRs in each chain are closely adjacent to the others by the FR regions and form an antigen-binding site of the nanobody with the CDRs of the other chain {see Kabat et al., 1991}.

As used herein, the term “heavy chain variable region” and “V_{H}” can be used interchangeably. As used herein, the terms “variable region” and “complementary determining region (CDR)” can be used interchangeably.

In another preferred teaching, the heavy chain variable region of said nanobody comprises 3 complementary determining regions: CDR1, CDR2, and CDR3.

The variable regions of the heavy chains of the nanobodies of the disclosure are of particular interest because at least a part of the variable region is involved in binding an antigen. Thus, the present disclosure includes those molecules having a nanobody heavy chain variable region with a CDR, provided that their CDRs, especially CDR3, are identical (or comprise at most one or two substitutions with respect to the identified CDR sequence) to the CDRs identified herein.

The nanobodies of the present invention may be humanised using techniques known to the skilled addressee (see, for example, {Vincke et al. 2009}).

The present teaching includes not only intact nanobodies but also antigen binding fragment(s) of an immunologically active nanobody or fusion protein(s) formed from a nanobody with other sequences. Therefore, the present disclosure also includes fragments, derivatives and analogs of the disclosed nanobodies.

As used herein, the terms “fragment,” “derivative,” and “analog” refers to a polypeptide that substantially retains the same biological function or activity of a nanobody of the invention. Polypeptide fragments, derivatives or analogs of the invention may be (i) polypeptides having one or more conservative or non-conservative amino acid residues substituted. Such substituted amino add residues may or may not be encoded by the genetic code; or (ii) a polypeptide having a substituent group in one or more amino add residues; or (iii) a polypeptide formed by fusing a mature polypeptide and another compound (such as a compound that increases the half-life of the polypeptide, for example, polyethylene glycol); or (iv) a polypeptide formed by fusing an additional amino acid sequence to the polypeptide sequence (e.g., a leader or secretory sequence or a sequence used to purify this polypeptide or a proprotein sequence, or a fusion protein formed with a 6 His tag). According to the teachings herein, these fragments, derivatives, and analogs are within the scope of one of ordinary skill in the art.

The nanobody of the present invention refers to a polypeptide including the above CDR regions having GPVI protein binding activity. The term also encompasses variant forms of polypeptides comprising the above CDR regions that have the same function as the nanobodies of the disclosure. These variations include, but are not limited to, deletion insertions and/or substitutions of one or several (usually 1-50, 1-30, 1-20, or 1-10) amino acids, and addition of one or several (generally less than 20, less than 10, or less than 5) amino acids at C-terminus and/or N-terminus. For example, in the art, the substitution of amino acids with analogical or similar properties usually does not alter the function of the protein. For another example, addition of one or several amino acids at the C-terminus and/or N-terminus may not change the function of the protein. The term also includes active fragments and active derivatives of the nanobodies of the disclosure.

The variant forms of the polypeptide include: homologous sequences, conservative variants, allelic variants, natural mutants, induced mutants, proteins encoded by DNAs capable of hybridizing with DNA encoding the nanobody of the present invention under high or low stringent conditions, and polypeptides or proteins obtained using antiserum against the nanobodies of the invention.

The disclosure also refers to other proteins or fusion expression products comprising a nanobody of the disclosure. Specifically, the present disclosure includes any protein or protein conjugate and fusion expression product (i.e. immunoconjugate and fusion expression product) having a heavy chain containing a variable region, of the nanobody of the present disclosure. As used herein, “conjugate,” “conjugation” or grammatical variations thereof refers the joining or linking together of two or more compounds resulting in the formation of another compound, by any joining or linking methods known in the art. A peptide linker/spacer sequence may also be employed to separate multiple polypeptide components by a distance sufficient to ensure that each polypeptide folds into its secondary and/or tertiary structures, if desired. Such a peptide linker sequence can be incorporated into a fusion polypeptide using standard techniques well known in the art.

In one teaching, a nanobody of the present invention may be conjugated or fused to a moiety which is capable of extending the half-life of the nanobody, as compared to the half-life of the nanobody that is not so conjugated to the moiety. In some embodiments, half-life is extended by greater than or greater than about 1.2-fold, 1.5-fold, 2.0-fold, 3.0-fold, 4.0-fold., 5.0-fold, or 6.0-fold. In some embodiments, half-life is extended by more than 6 hours, more than 12 hours, more than 24 hours, more than 48 hours, more than 72 hours, more than 96 hours or more than 1 week after in vivo administration compared to the nanobody without the half-life extending moiety. The half-life refers to the amount of time it takes for the protein to lose half of its concentration, amount, or activity. Half-life can be determined for example, by using an ELISA assay or an activity assay. Exemplary half-life extending moieties include an Fc domain, a multimerization binding domain (e.g. albumin binding domain), human serum albumin (HSA), or bovine serum albumin (BSA).

An Fc (fragment crystallizable) region or domain of an immunoglobulin molecule (also termed an Fc polypeptide) corresponds largely to the constant region of the immunoglobulin heavy chain, and is responsible for various functions, including the antibody's effector function(s). The Fc domain contains part or all of a hinge domain of an immunoglobulin molecule plus a CH2 and a CH3 domain. The Fc domain can form a dimer of two polypeptide chains joined by one or more disulfide bonds.

The half-life extending moiety may be directly fused or conjugated to the nanobody, or a linker may be employed. For example, any half-life extending moiety, such as a Fc region may be linked indirectly or directly to one or more nanobodies or antigen binding fragments thereof. Various linkers are known in the art and can optionally be used to link an Fc (or other half-life extending moiety) to a nanobody to generate an Fc-fusion. Fc-fusions of identical species can be dimerized to form Fc-fusion homodimers, or using non-identical species to form Fc-fusion heterodimers.

As well as those identified above, as known by those skilled in the art, immunoconjugates and fusion expression products include: conjugates formed by binding drugs, toxins, cytokines, radionuclides, enzymes, and other diagnostic or therapeutic molecules to the nanobodies or fragments thereof of the present invention.

The disclosure also provides other polypeptides, such as a fusion protein comprising a nanobody or antigen binding fragment thereof. In addition to almost full-length polypeptides, the present invention also includes fragments of the nanobodies of the disclosure. Typically, the fragment has at least about 50 contiguous amino acids of a nanobody described herein, such as at least about 50 contiguous amino acids, at least about 80 contiguous amino acids, or at least about 100 contiguous amino acids. The fragment may comprise at least the CDR3 region of a nanobody as described herein.

In the present teaching, “a conservative substitution” refers to a polypeptide in which there are up to 10, up to 8, up to 5, or up to 3 amino acids substituted by amino acids having analogical or similar properties, compared to the amino acid sequence of the nanobody from which the polypeptide is derived. These conservative variant polypeptides may be produced according to the following amino acid substitutions:

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

The present teaching also provides a polynucleotide molecule encoding a nanobody or antigen binding fragment or fusion protein thereof. Polynucleotides of the invention may be in the form of DNA or RNA. DNA forms include cDNA, genomic DNA, or synthetic DNA. DNA can be single-stranded or double-stranded. DNA can be a coding strand or a non-coding strand. DNA sequences encoding the nanobodies according to SEQ ID NOS: 1-54 are provided in corresponding SEQ ID NOS: 55-108. Thus, the nanobody defined by SEQ ID NO:1 is encoded by SEQ ID NO:55, the nanobody defined by SEQ ID NO:2 is encoded by SEQ ID NO:56 and so on.

Polynucleotides encoding the nanobodies of the disclosure include: coding sequences only encoding a nanobody; and coding sequences for the nanobody and various additional coding sequences; coding sequences (and optional additional coding sequences).

The term “polynucleotide encoding a polypeptide” may include a polynucleotide that encodes the polypeptide, and may also include a polynucleotide that includes additional coding and/or non-coding sequences.

The full-length nucleotide sequence of the nanobody of the present disclosure or an antigen binding fragment thereof can generally be obtained by a PCR amplification method, a recombination method, or an artificial synthesis method. One possible method is to synthesize related sequences using synthetic methods, especially when the fragment length is short. In general, a long sequence of fragments can be obtained by first synthesizing a plurality of small fragments and then connecting them. In addition, the coding sequence of the heavy chain and an expression tag (e.g. 6 His) can be fused together to form a fusion protein.

Once the desired sequences have been obtained, the desired sequences can be obtained in large scale using recombinant methods. Usually, sequences can be obtained by cloning a desired polynucleotide into a vector, transforming or transfecting the vector into cells, and then isolating the sequences from the proliferated host cells by conventional methods. Bio-molecules (nucleic acids, proteins, etc.) to which the present disclosure relates include bio-molecules that exist in isolated form.

Nucleic acid sequences encoding a nanobody of the present disclosure (or an antigen binding fragment thereof, or a derivative thereof) can be obtained completely by chemical synthesis. The DNA sequence then can be introduced into various existing DNA molecules (or e.g. vectors) and cells known in the art. In addition, mutations can also be introduced into the protein sequences of the disclosure by chemical synthesis.

The disclosure also relates to vectors comprising the above-mentioned suitable DNA sequences and suitable promoters or control sequences. These vectors can be used to transform an appropriate host cell so that it can express the protein.

The host cell can be a prokaryotic cell, such as a bacterial cell; or a lower eukaryotic cell, such as a yeast cell; or a higher eukaryotic cell, such as a mammalian cell. Representative examples are: Escherichia coil, Streptomyces, bacterial cells such as Salmonella typhimurium, fungal cells such as yeast, insect cells of Drosophila S2 or Sf9, animal cells of CHO, COST, 293 cells, and the like.

The transformation of the host cell with the recombinant DNA can be performed using conventional techniques well known to those skilled in the art. When the host is a prokaryotic organism such as E. coli, competent cells capable of absorbing DNA can be harvested after the exponential growth phase and treated with the CaCl₂ method. The procedures used are well known in the art. Another method is to use MgCl₂. If necessary, conversion can also be performed by electroporation. When the host is eukaryotic, the following DNA transfection methods can be used: calcium phosphate coprecipitation, conventional mechanical methods such as microinjection, electroporation, liposome packaging, and the like.

The obtained transformants can be cultured in a conventional manner to express the polypeptide of interest. Depending on the host cells used, the medium used in the culture may be selected from various conventional media. The culture is performed under conditions suitable for the host cells growth. After the host cells are grown to an appropriate cell density, the selected promoter is induced by a suitable method (such as temperature shift or chemical induction) and the cells are incubated for a further period of time.

The recombinant polypeptide in the above method may be expressed intracellularly, or on the cell membrane, or secreted extracellularly. If necessary, the recombinant protein can be isolated and purified by various separation methods by utilizing its physical, chemical and other characteristics. These methods are well-known to those skilled in the art. Examples of these methods include, but are not limited to: conventional renaturation treatment, treatment with a protein precipitation agent (salting out method), centrifugation, osmotic disruption, super treatment, ultracentrifugation, molecular sieve chromatography (gel filtration), adsorption layer analysis, ion exchange chromatography, high performance liquid chromatography (HPLC), and various other liquid chromatography techniques and the combinations thereof.

The nanobodies of the disclosure may be used alone or in combination or conjugated with, for example, a detectable marker (for diagnostic purposes), a therapeutic agent, modification moiety, or a combination thereof.

Detectable markers for diagnostic purposes include, but are not limited to: fluorescent or luminescent markers, radioactive markers, MRI (magnetic resonance imaging) or CT (computed tomography) contrast agents, or enzymes capable of producing detectable products.

The disclosure also relates to the use of the nanobodies, antigen binding fragments and derivatives/fusions, as described herein, for use in a method of treating or preventing diseases arising from processes of blood platelet aggregation, as well as other related conditions.

The nanobodies, antigen binding fragments and derivatives/fusions, as described herein may be used in methods for the prevention and treatment of thrombosis. The present disclosure also relates to in vitro screening methods for identifying an inhibitor of GPVI mediated adhesion of platelets to active intravascular lesions.

The nanobodies, antigen binding fragments and derivatives/fusions, as described herein may be used in methods for the treatment and/or prevention of acute and chronic vascular diseases associated with intraarterial and/or intravenous thrombosis, such as acute coronary syndrome/acute myocardial infarction, chronic coronary syndrome/stable coronary artery disease, transient ischaemic attack, stroke, peripheral vascular disease, deep vein thrombosis, thromboprophylaxis in patients with atrial fibrillation, thromboprophylaxis in patients with recent medical or surgical admission to hospital, pulmonary embolism, sepsis-related coagulopathy, COVID-19 associated thrombosis, thrombosis related to cardiac devices (e.g. left ventricular assist devices, extra extracorporeal membrane oxygenation and mechanical heart valves), left ventricular thrombosis, thromboprophylaxis in heart failure, thromboprophylaxis in thrombophilic conditions (e.g. antiphospholipid syndrome, factor V Leiden) and other forms of inflammation, infection and cancer-driven thrombosis.

The inhibition of platelet activation leads to a general impairment of the platelets with regard to their ability to aggregate, adhere to surfaces, release their granule contents and generate thromboxanes and other lipid species.

Moreover, the present disclosure aims to provide an inhibitor of GPVI, notably human GPVI, which does not activate the GPVI receptor by intrinsic antibody activity and which does not induce immuno-thrombocytopenia. Moreover, the present disclosure aims to provide an inhibitor for the release mechanism of platelets and the expression of pro-inflammatory responses.

A nanobody of the present disclosure, or antigen-binding fragment or variant thereof, may be administered in combination with another therapeutic agent such as antiplatelet drugs, including aspirin (and other cyclooxygenase inhibitors), P2Y₁₂ receptor antagonists and GPIIb/IIIa inhibitors, as well as anticoagulants including heparin, warfarin and direct inhibitors of Factor Xa and thrombin, and thrombolytic therapy such as reteplase, alteplase and streptokinase.

Where a nanobody of the present disclosure, or antigen-binding fragment or variant thereof is administered in combination therapy with one, two, three, four or more, preferably one or two, preferably one other therapeutic agent, the nanobody and agent(s) can be administered simultaneously or sequentially. When administered sequentially, they can be administered at closely spaced intervals (for example over a period of 5-10 minutes) or at longer intervals (for example 1, 2, 3, 4 or more hours apart, or even longer period apart where required), the precise dosage regimen being commensurate with the properties of the therapeutic agent(s).

The nanobodies of the invention may also be administered in conjunction with non-active agent treatments such as, photodynamic therapy, gene therapy; or surgery.

The subject is typically a human.

The nanobodies will generally be administered in a therapeutically or prophylactically effective amount. By a therapeutically or prophylactically effective amount is meant one capable of achieving the desired response, and will be adjudged, typically, by a medical practitioner. The amount required will depend upon one or more of at least the active compound(s) concerned, the patient, the condition it is desired to treat or prevent and the formulation of order of from 1 μg to 1 g of compound per kg of body weight of the patient being treated.

Different dosing regimens may likewise be administered, again typically at the discretion of the medical practitioner. Nanobodies of the disclosure, may be provided by daily administration although regimes where the compound(s) is (or are) administered more infrequently, e.g. every other day, weekly or fortnightly, for example, are also embraced by the present disclosure.

By treatment is meant herein at least an amelioration of a condition suffered by a patient; the treatment need not be curative (i.e. resulting in obviation of the condition). Analogously references herein to prevention or prophylaxis herein do not indicate or require complete prevention of a condition; its manifestation may instead be reduced or delayed via prophylaxis or prevention according to the present disclosure.

The compounds of the present disclosure may be purchased from commercial suppliers, or prepared using reagents and techniques readily available in the art.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation.

Preferably, a pharmaceutical composition is suitable for intravenous, intramuscular, subcutaneous, parenteral, spinal or epidermal administration (e.g., by injection or infusion). Depending on the route of administration, the active nanobody can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a nanobody of the invention can be administered via a non-parenteral route, such as a topical, epidermal or mucosal route of administration, e.g., intranasally, orally, vaginally, rectally, sublingually or topically.

The nanobodies of the invention can be in the form of pharmaceutically acceptable salts. A “pharmaceutically acceptable salt” refers to a salt that retains the desired biological activity of the parent nanobody and does not impart any undesired toxicological effects. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Pharmaceutical compositions can be in the form of sterile aqueous solutions or dispersions. They can also be formulated in a microemulsion, liposome, or other ordered structure suitable to high drug concentration. The amount of active ingredient, which can be combined with a carrier material to produce a single dosage form will vary depending upon the subject being treated and the particular mode of administration and will generally be that amount of the composition, which produces a therapeutic effect. Generally, out of one hundred percent, this amount will range from about 0.01% to about ninety-nine percent of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, antibody can be administered as a sustained release formulation, in which case less frequent administration is required.

For administration of the nanobody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for a nanobody of the disclosure include 1 mg/kg body weight or 3 mg/kg body weight via intravenous administration, with the nanobody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 mg/ml and in some methods about 25-300 mg/ml.

In some embodiments, nanobodies in accordance with the present invention, such as the nanobody according to SEQ ID NO:28, encoded by SED ID NO: 82, do not compete with other described nanobodies, such as Nb2 and Nb35, for binding to GPVI. Such antibodies may find use in imaging studies of GPVI, particularly when conjugated to a tag molecule, such as a fluorescent molecule as described herein.

### DETAILED DESCRIPTION

The present disclosure will be further described with reference to the following figures, which show:

FIG. 1. Testing of nanobody binding and inhibition of GPVI signalling. a) NFAT reporter assay of GPVI and FcRγ transfected DT40 cells stimulated by collagen (10 μg/ml) in the presence of the nanobodies (100 nM). Results were plotted as a percentage of total signalling in the presence of collagen only. Dotted lines represent 100, 60 and 20% signalling levels. Data represent mean values of three experiments performed in triplicate±standard deviation. b) Surface binding assay of one nanobody (100 nM) from each family binding to a GPVI-Fc coated-surface. All the binding results have been normalised to Nb35 which gave the highest readout. Binding of each nanobody to the Fc domain-coated surface was tested and subtracted from the GPVI-Fc readings. Binding was detected using HRP conjugated anti-His antibody. The average binding of all the nanobodies to BSA represents a non-specific binding control. Data represent mean values of three experiments±standard deviation.

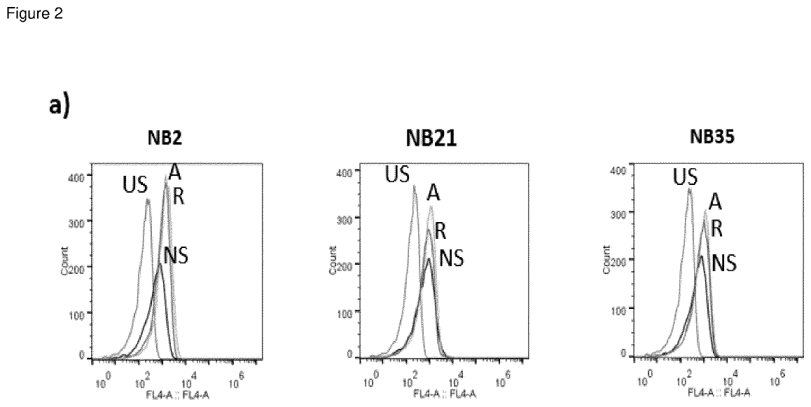

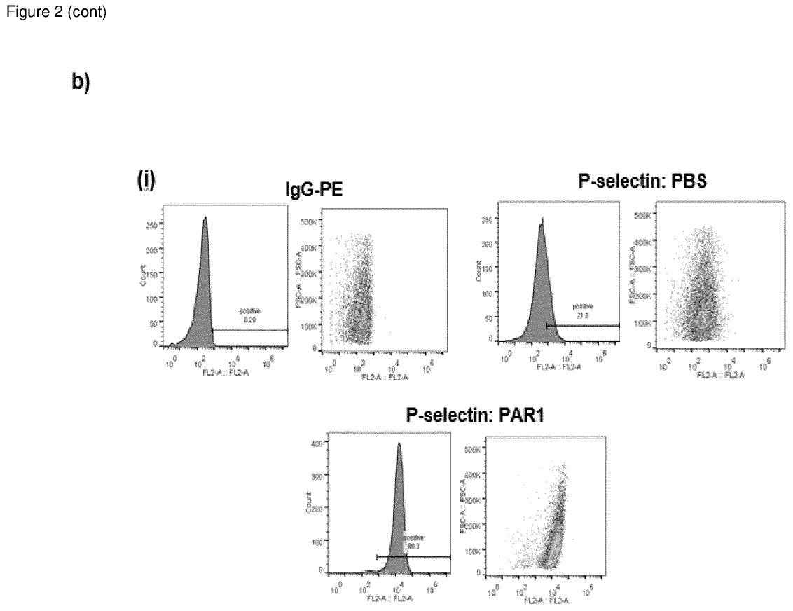

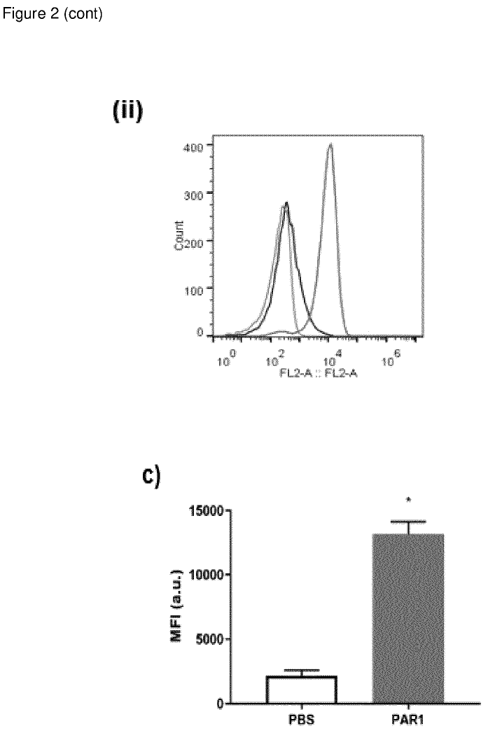

FIG. 2. Nanobodies 2, 21 and 35 binding to washed platelets. a) Nanobodies 2, 21 and 35 (5 μM) binding to washed platelets in the presence or absence of 200 μM PAR1 by flow cytometry. Histograms represent unstained washed platelets (US), non-specific platelet staining with anti-his alexafluor647 secondary antibody (NS), nanobody binding to resting platelets (R) and nanobody binding to PAR1 peptide-activated platelets (A). b) Flow cytometry data showing 10,000 events/sample of PAR-1 (200 μM) activated platelets vs resting platelets using PE-conjugated anti-human CD62P antibody or corresponding isotype-matched control (IgG1 PE antibody). i-ii) Representative raw fluorescent intensity histograms and dot blots, and histogram overlays respectively. c) Flow cytometry data comparing P-selectin expression in resting, PAR-1 activated platelets. Significance was measured using a two-tailed Student t-test where P=≤0.05. Data represents mean values±SEM (n=7).

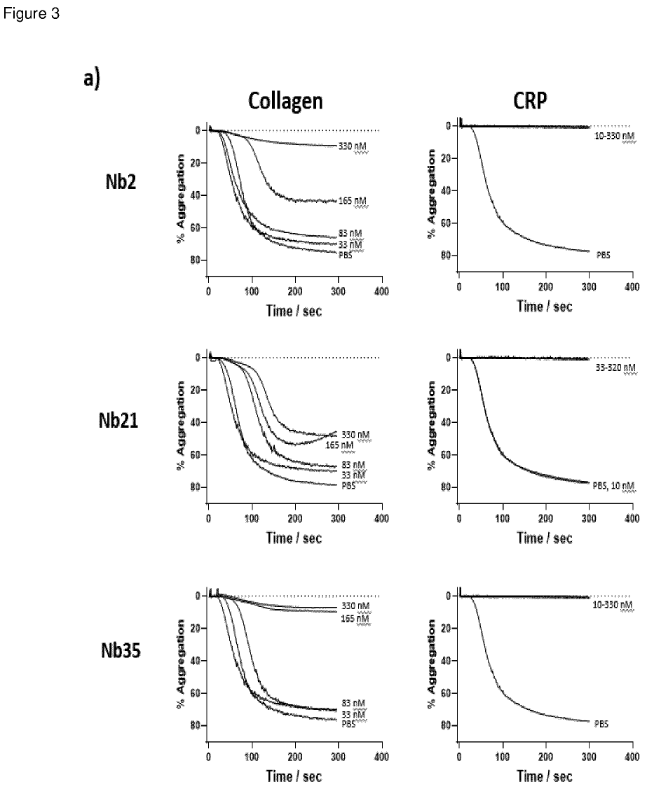

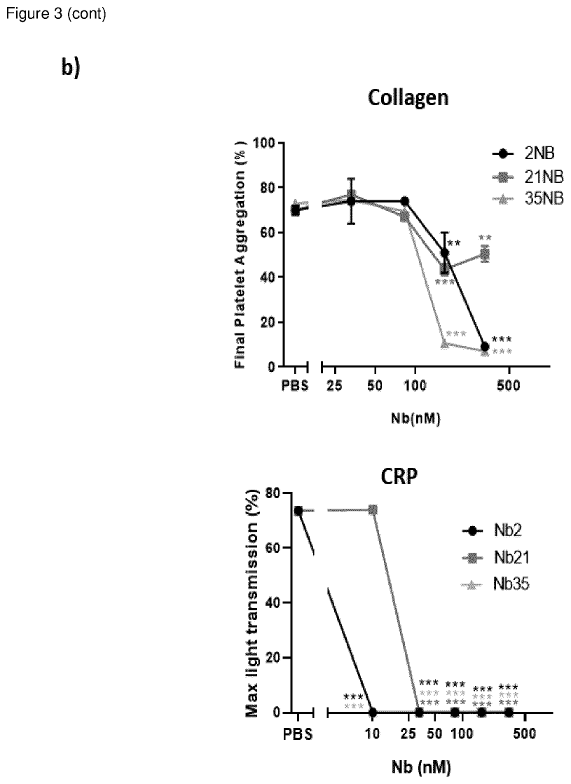

FIG. 3. Nanobodies 2, 21 and 35 inhibit aggregation in response to collagen and CRP. Washed platelet aggregation stimulated by 5 μg/ml collagen or 10 μg/ml CRP in the presence of increasing concentrations of Nb2, 21 and 35. Raw aggregation curves a) and max aggregation plots b) are shown. IC50 values of 172, 85 and 115 nM for collagen and 1, 22 and 1 nM for CRP were determined for Nb2, 21 and 35, respectively. The effect of different concentrations of the nanobody compared the vehicle (PBS) was determined using two-way ANOVA with Dunnett's correction for multiple comparisons.

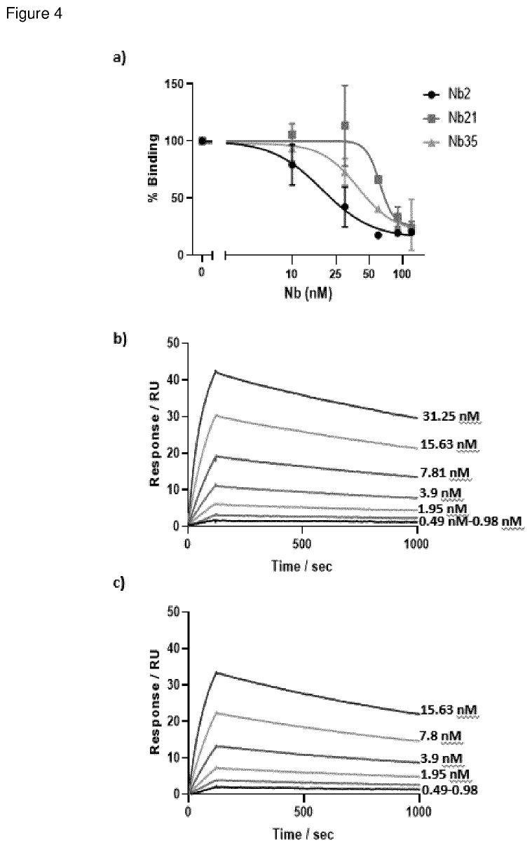

FIG. 4. Nanobodies 2, 21 and 35 inhibit collagen binding, and Nb2 binds GPVI with nanomolar affinity. a) Solid-phase binding assay showing GPVI-Fc (100 nM) displacement from a collagen surface in the presence of increasing concentration Nb2, 21 and 35, with IC50 values of 18, 62 and 39 nM respectively. Data represent mean values of three±standard deviation. b-c) SPR data showing Nb2 at a range of concentrations binding to GPVI-Fc (b) and GPVI (c) immobilised on a surface. The binding affinity was determined by kinetic analysis with calculated Kd values of 0.7 nM±0.03 nM for GPVI-Fc and 0.58 nM±0.06 nM for GPVI.

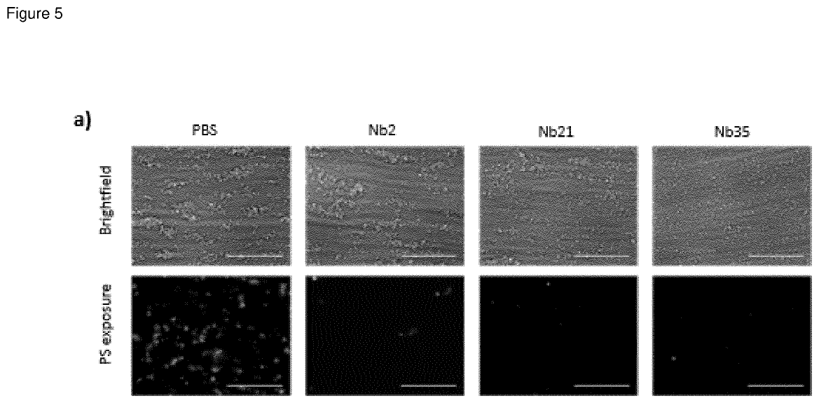

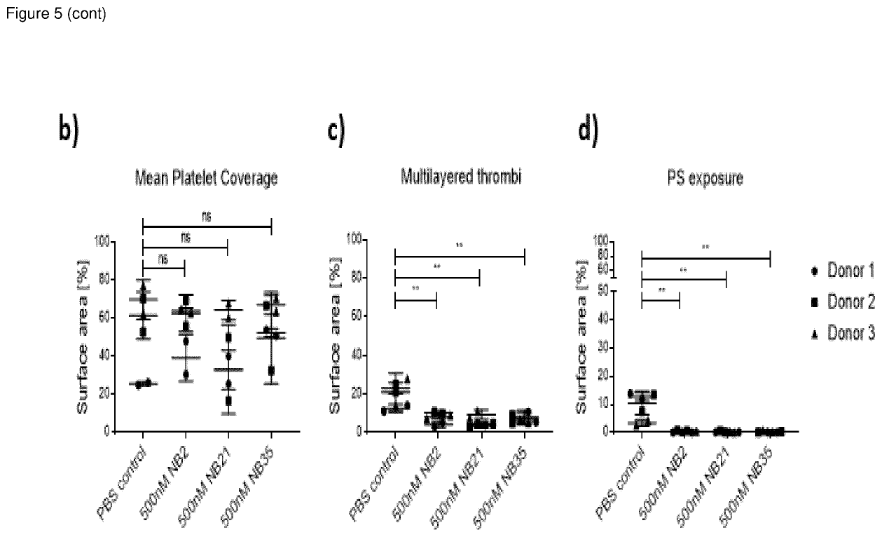

FIG. 5. Effect of anti-GPVI nanobodies in whole blood microfluidics. a) Representative images of whole blood perfused at an arterial shear rate (1000/s) over Horm Collagen I in the presence of either PBS, 500 nM nanobody 2, 21 or 35. Adhered platelets and platelet aggregates were imaged in brightfield after 3.5 minutes of flow. Platelets were labelled with Annexin V AF568 to assess phosphatidylserine (PS) exposure. All images were taken on an EVOS AM F4300 using a 60×, 1.42NA oil objective. Data are representative of 2 runs for each of 3 donors per treatment. Scale bar: 50 μm. Quantitative analysis of the images assessed the effect of the nanobodies on percentage of total surface area covered by b) platelets, c) multilayered thrombi and d) platelets exposing PS. Data points are individual runs for each of 3 donors (shown in different colours) per treatment (Mean±SD). Unpaired t-test with Mann Whitney correction was employed to test for statistical significance, ** p<0.005. SAC=Surface area coverage.

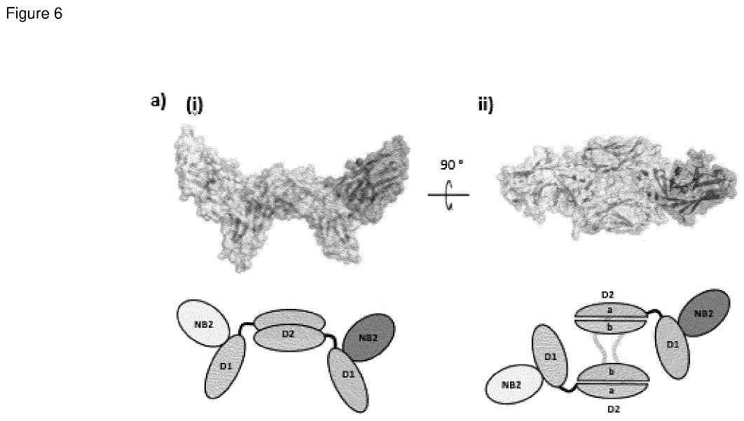

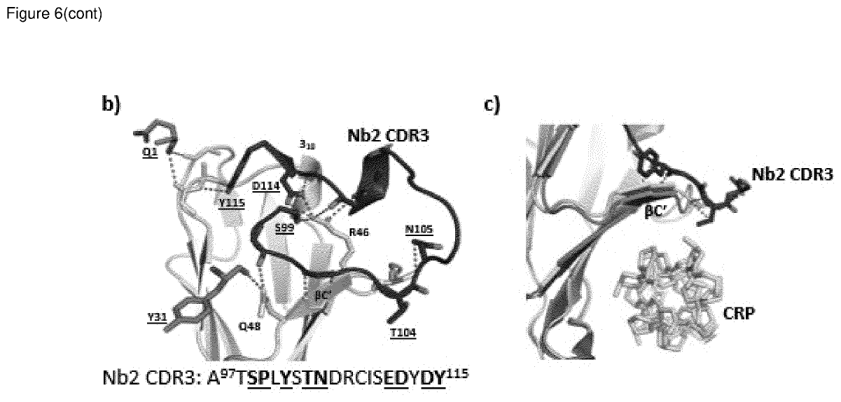

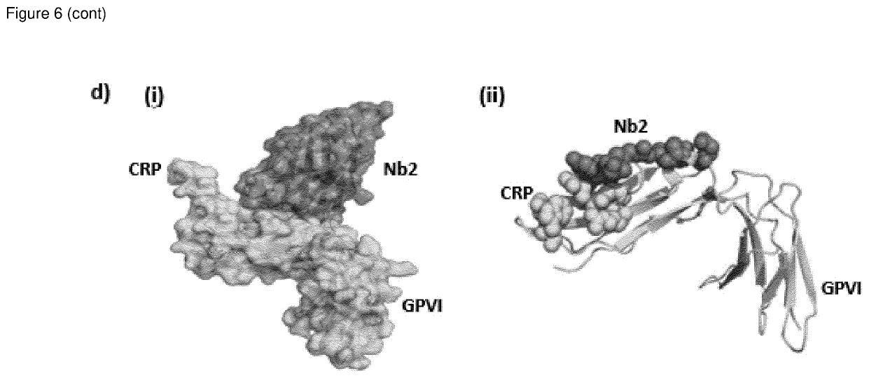

FIG. 6. Site of interaction of GPVI with Nb2 and CRP. a) Structure of Nb2 binding to GPVI. (i) A side view of the GPVI-Nb2 structure; (ii) A top down view illustrating the domain swap hinge region. A cartoon representation is shown below each figure. The domain-swapped D2 domains are labelled D2a and D2b for the N and C-terminal D2 regions respectively. b) Zoomed in view of the GPVI : Nb2 binding interface. Dashed lines indicate polar contacts made between GPVI and Nb2 residues, and residues from Nb2 are underlined to distinguish them from GPVI residues. The full CDR3 loop region is provided underneath the structure with binding residues highlighted in bold. c) Zoomed in view of the CRP binding groove of the CRP bound GPVI structure and nanobody bound structure. The binding of Nb2, largely through the CDR3 loop, towards the top of the binding groove results in a shift of the βC′ sheet resulting in a small distortion of the CRP binding groove. d) Locations of the known Nb2 and CRP binding sites on GPVI. i) Surface representation of Nb2 modelled onto the GPVI-CRP complex structure (PDB: 5OU8) revealing the two non-overlapping but closely situated binding sites. ii) Nb2 and CRP binding residues mapped on the structure of GPVI.

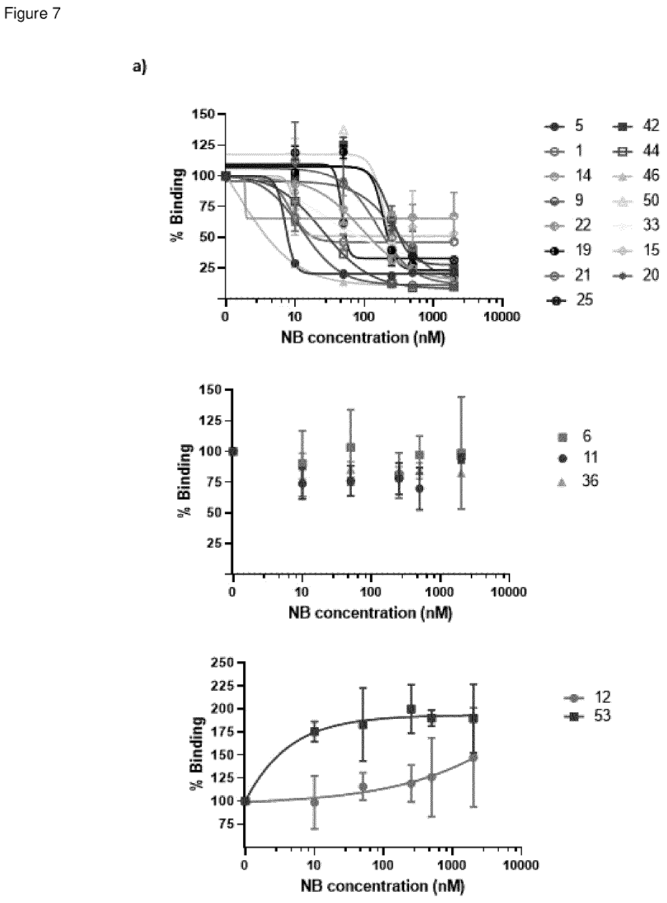

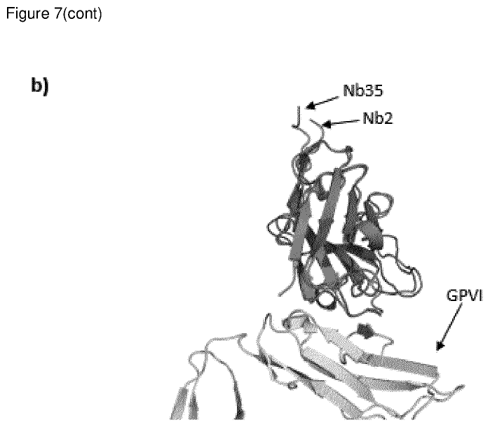

FIG. 7. Nb2 shares overlapping binding sites to other nanobodies. A) His-tagged nanobodies (100 nM) binding to a GPVI-Fc (10 nM) immobilized surface in the presence of increasing concentration of un-tagged Nb2. The majority of the nanobodies were displaced in the presence of Nb2 (top panel), no displacement was detected with nanobodies 6, 11, 14 and 36 (middle panel), and nanobody 2 increased the binding of both nanobodies 12 and 53 (bottom panel). B) Nb2 and Nb35 binding sites overlayed on GPVI as determined by x-ray crystallography. The Nb2 and Nb35 binding sites directly overlap with one another.

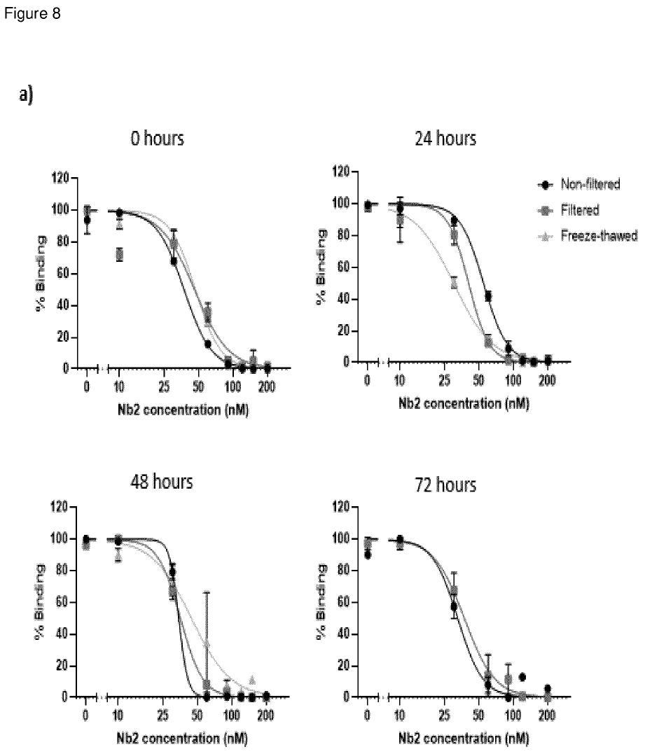

FIG. 8. Nb2 stability assay. Nb2 stability over 72 hours at 4° C., and after syringe filtering or freeze-thawing, as determined by the displacement of GPVI-Fc (100 nM) binding to a collagen coated surface. Nb2 was tested at 0-, 24-, 48- and 72-hours.

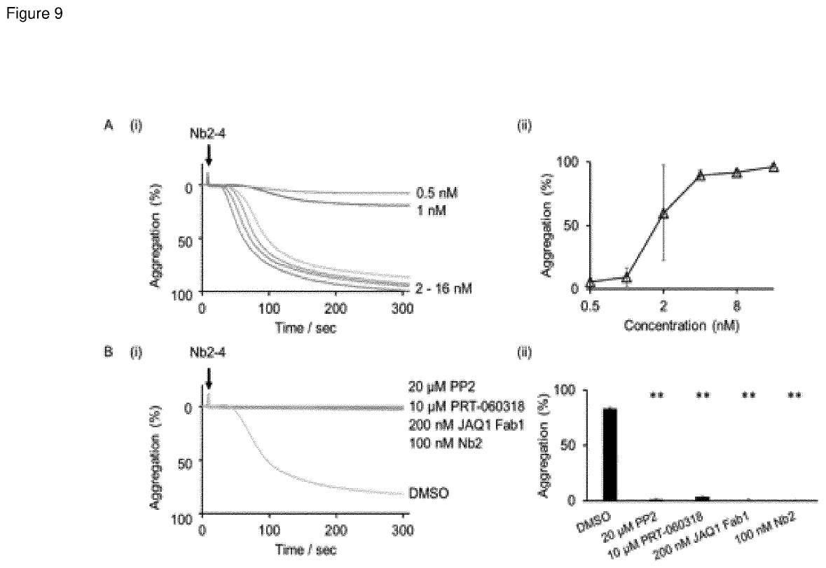

FIG. 9. Tetravalent Nb2 (Nb2-4) stimulates platelet aggregation

(A) Washed platelets at 2×10⁸/ml isolated from healthy donors were stimulated with tetrameric Nb2 (Nb2-4) generated by crosslinking four Nb2 using a (GGGGS)₃ linker. (B) Washed platelets at 2×10⁸/ml were incubated with vehicle, PP2 (20 μM), PRT-060318 (10 μM), JAQ1 Fab1 (200 nM) or Nb2 (100 nM) for 10 minutes, before stimulation with 16 nM Nb2-4. LTA was monitored for 5 minutes. (i) Representative traces of three identical aggregation experiments. (ii) Mean±SD (%) aggregation after 5 minute of agonist stimulation. **(P<0.01) calculated using Student's paired t-test. N=3 separate donors.

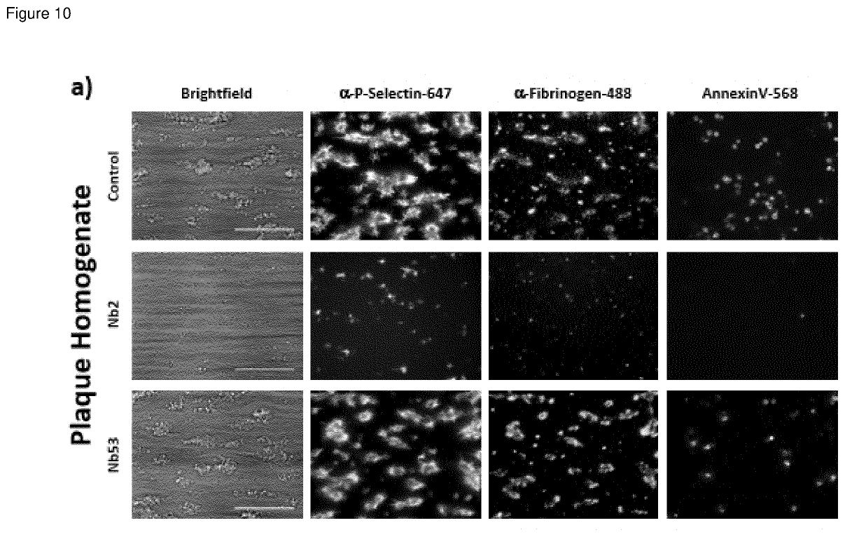

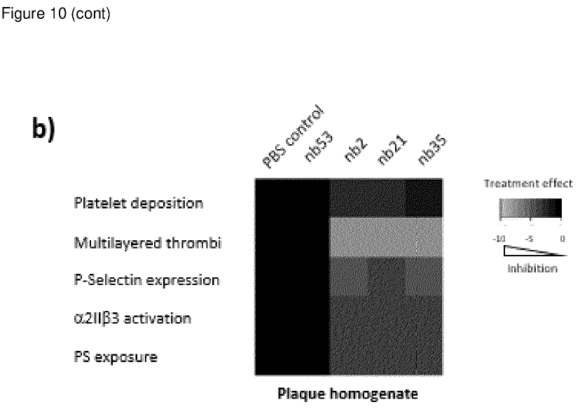

FIG. 10. Nbs 2, 21 and 35 inhibit thrombus formation on atherosclerotic plaque at arterial shear rates.

Blood was preincubated with either vehicle (PBS), 500 nM Nb2, Nb21, Nb35 or Nb53 (negative control) for 10 minutes and then perfused at 1000/s over plaque homogenate for 3.5 min. Brightfield images give information about thrombus size and morphology; P-Selectin is used to assess α-granule secretion, anti-fibrinogen to indicate αIIbβ3 activation and Annexin V to approximate procoagulant activity by PS exposure. (a) Representative images of thrombi formed on pooled plaque homogenate in the presence of Nb2 or negative control Nb53. Scale bars=50 μM. (b) Heatmaps of all Nb treatments, generated by subtracting the scaled value of the vehicle from the scaled value of the respective treatment and parameter; then filtered for significance. Nbs 2, 21 and 35 significantly inhibited all tested parameters. Colour-code: lighter squares indicate greater significance. n=5-8, tested for p<0.05 with a non-parametric one-way ANOVA

FIG. 11. Nb2 strongly inhibits plaque-mediated GPVI signalling.

(a) Western blot analysis of phosphorylation status of total protein and the indicated GPVI signalling cascade proteins and the loading control total Syk, following platelet stimulation with fibrillar collagen I or plaque for 180 seconds in the presence or absence of Nb2 or Nb53. (b) Quantitation of phospho-proteins from n=3 experiments shows strong inhibition of GPVI signalling by Nb2. Graphs: Mean±SD, non-parametric one-way AVOVA, *P<0.05, **P<0.005.

FIG. 12. Nb 28 does not affect aggregation, adhesion and activation under flow or compete with Nb2 binding.

a) Washed platelets were incubated in an aggregometer at 37° C. and 1200 rpm in the presence of Nb28 or PBS control prior to stimulation with 10 μg/ml Horm collagen and aggregation traces captured. Nb28 had no effect on aggregation. b) Thrombin inhibited whole blood was preincubated with either vehicle (PBS) or 500 nM of unlabelled Nb28 and then flowed over fibrillar collagen I utilising the Maastricht flow chamber at 1000/s. None of the five investigated parameters of thrombus formation or platelet activation were affected by Nb28. Each data point represents one donor (n=4), unpaired non-parametric t-test was utilized to test for significance. To test for competitive binding between Nb28 and Nb2, washed platelets were preincubated 500 nM Nb2 or vehicle (PBS) for 10 minutes, then labelled with 100 nM Nb28-AF647 and assessed for fluorescence by flow cytometry with 50,000 events acquired. c) Representative flow cytometry frequency plot. d) Quantitative analysis (mean+/−SD; n=3) showed preincubation with 500 nM Nb2 for 10 min did not affect Nb28-647 binding to washed platelets (non-parametric two-way ANOVA, ns=not significant; **** P<0.0001).

FIG. 13. Fluorescent imaging of labelled Nb28 reveals Nb2 disrupts GPVI clustering along collagen under flow.

a) In a whole blood flow assay (1000 s⁻¹) GPVI, labelled with 100 nM Nb28-AF647, formed clearly visible clusters along the fibrillar collagen (arrows). B) Preincubation of platelets with 500 nM Nb2 disrupted the clustering. Arrows indicate areas where collagen fibres are visible. n=5-7, Scale bars=10 μM.

### METHODS

### Materials

The expression plasmid for recombinant GPVI-Fcγ (GPVI residues 22-203) in the Sigplg⁺ plasmid as previously reported {Onselaer, 2017}. Nanobodies were raised against GPVI through VIB Nanobody core (VIB Nanobody Service Facility, Brussels, https://corefacilities.vib.be/nsf) and the DNA sequences were provided in PMECS vector. Goat anti-human immunoglobulin G (IgG) and rabbit anti-6-His HRP antibodies were purchased from ThermoFisher Scientific (Glasgow, United Kingdom) and Cambridge Bioscience (Cambridge, United Kingdom) respectively. Alexa Fluor-647 rabbit anti-6-His and Alexa Fluor-647 dye for Nb labelling were purchased from ThermoFisher Scientific (Paisley, UK). Collagen was purchased from Nicomed and collagen related peptide (CRP) was prepared as previously described {Raynal, 2006}. CD62P-PE and isotype IgG1 κ-PE were from Biolegend (San Diego, Ca, USA). CRP-XL was purchased from CAMBOL laboratories (Cambridge, UK). PAR1 activating peptide (SFLLRN) was purchased from Severn Biotech (Kidderminster, UK). Atherosclerotic plaque material was prepared by homogenising plaques removed from 10 patients by carotid endarterectomy into PBS and pooling the material.

### PCR Mutagenesis

Site-directed mutagenesis was performed on GPVI to produce the glycosylation (N72/Q) and domain swap hinge deletion mutants, and thrombin cleavable nanobodies. All mutagenesis was performed using a Q5 site-directed mutagenesis kit (New England Biolabs) following the provided protocol. The primers used are shown below:

| | | | Ta |
|---|---|---|---|
| Construct | | Primers | (° C.) |
| GPVI N72/Q | | F: | 69 |
| | | CTCCTACCAGcag | |
| | | GGAAGCCTCTGGTC | |
| | | R: | |
| | | CAGCGGTAGCGTC | |
| | | CAGCC | |
| Nanobody | thrombin | F: | 70 |
| insertion | | cgcggcagcGCGGC | |
| | | CGCATACCCGTAC | |
| | | R: | |
| | | cggcaccagTGAGG | |
| | | AGACGGTGACCTGG | |

Primer sequences and annealing temperatures (Ta) used for all the mutagenesis studies.

### Expression and Purification of Recombinant GPVI

GPVI-Fc (dimeric) was expressed and purified in the Sigplg expression vector as previously described {Onselaer, 2017}. The construct consists of both the D1 and D2 domains (residues 1-183), but does not contain the stalk like other GPVI-Fc constructs including Revacept. The construct consists of both the D1 and D2 domains (residues 1-183), but does not contain the stalk. Monomeric GPVI was produced by cleavage of the Fc domain by incubating with human Factor Xa for 12-18 hours at room temperature (1 μg FXa for every 250 μg of GPVI) in the presence of 2.5 mM CaCl₂. Protein-A chromatography was used to separate the cleaved Fc and GPVI followed by gel filtration using a Superdex 75 26/60. All proteins were snap frozen and stored at −80° C.

### Expression and Purification of GPVI Nanobodies.

All nanobodies were expressed in E. coli wk6 cells grown in 1 L TB media with 100 μg/ml ampicillin. Cells were left to grow at 37° C. with 180 rpm shaking and once grown to an OD of 0.6-0.9 Au, the cells were induced with 1 mM isopropyl β-d-1-thiogalactopyranoside. Cells were incubated for 18 hours at 28° C. and cell pellets were collected by centrifuging at 3,000×g for 20 min. 1 L of pellet was re-suspended in 12 mL TE buffer (0.2 M Tris pH 8.0, 0.5 mM EDTA, 0.5 M sucrose) and incubated at 4° C. for 1 hour. A further 18 mL of TE diluted 4-fold in water was added and incubated for 1 hour at 4° C. The re-suspended cells were centrifuged at 8,800×g for 30 min, and the supernatant was incubated with 1 mL His-pure Ni-NTA resin (ThermoFisher) for 20 min at room temperature. The beads were washed with 20 mL PBS and the tagged-nanobody was eluted with 2 mL 500 mM imidazole and dialysed into PBS. For the tag-cleavable nanobodies, the tags were cleaved by incubating with 10 U of human α-thrombin and 2.5 mM CaCl₂ for 18 hours at room temperature. The cleaved nanobodies were subject to gel filtration using Superdex 75 26/60 equilibrated in PBS. Nanobodies were snap frozen and stored at −80° C.

### Generation of Divalent and Tetravalent Nb2 (Nb2-4)

Divalent Nb2 (Nb2-2) and tetravalent Nb2 (Nb2-4) were generated by linking individual Nb2 domains with a (GGGGS)₃ linker. Nb2-2 was generated in a two step process. Firstly, XmaI and SaII restirction sites were inserted into the regular Nb2 plasmid, just before the HA tag, using the following primers and an annealing temperature of 70° C.:

F: GTCGACGCGGCCGCATACCCGTAC
R: CCCGGGTGAGGAGACGGTGACCTGG

The second step involved the purchase of the entire Nb2 gene with 5′ XmaI and 3′ SaII resitrciton sites. This gene was purchased from TwistBiosciences. Both constructs were restriction digested and ligated together resulting in a Nb2 dimer in the original plasmid with C-terminal HA and His tags. The Nb2-4 gene was synthesized by VIB NanobodyCore and supplied in a pHEN6c expression plasmid with the same C-terminal tags as the other nanobodies. Expression and purification of these constructs was identical to that of the regular nanobodies.

### Solid-Phase Binding Assay

Solid-phase binding assays were performed following previously documented experimental protocols {Onselaer, 2017}. Wells were coated with collagen (4 μg/ml) or CRP (4 μg/ml) and the binding of GPVI-Fc (100 nM) was detected in the presence of increasing concentrations of nanobody. HRP-conjugated anti-Fc antibody was used for the detection of GPVI-Fc binding.

### Flow Cytometry

Washed platelets (2×10⁸/ml) were diluted (1:10) in PBS and incubated with PAR1-activating peptide (200 μM) or vehicle for 3 min at room temp. To determine platelet P-selectin expression, a sample of each condition was incubated with a CD62P-PE or isotype IgG1 κ-PE antibody for 20 min at room temp. The unstimulated and stimulated platelets were incubated with the nanobodies (5 μM) for 30 min at room temp followed by Alexa Fluor-647 rabbit anti-6-His antibody secondary labelling (1:80). Unstimulated control samples with no staining and secondary antibody staining only were made and analysed. The samples were acquired (FL2 and 4) and analysed in an Accuri C6 flow cytometer (BD Biosciences, USA) as previously described {Nagy et al 2020}.Histograms were made in FlowJo v10.0.7 (Eugene, OR).

### Flow Cytometry Competition Assay

Washed platelets were preincubated with 500 nM Nb2 or PBS vehicle control for 10 min, followed by addition of 100 nM Nb28-AF647 and immediate fixation. 50,000 events were acquired and Nb28-AF647 signal (FL4) measured with an Accuri C6 flow cytometer (BD) and data processed with the BD Accuri C6 Software.

### Platelet Aggregation Assay

Human platelets were freshly prepared and aggregation measured as previously described {Onselaer, 2017}. Platelets were incubated with the nanobody for 10 min before stimulation with 5 μg/mL collagen or 10 μg/mL CRP-XL. The effect of different concentrations of the nanobody compared to PBS was determined using two-way ANOVA with Dunnett's correction for multiple comparisons

### Whole Blood Microfluidics

Degreased glass coverslips were coated with 0.5 μl microspots of 100 μg/ml Horm Collagen I (diluted in manufacturer supplied diluent) or 500 μg/ml pooled plaque homogenate overnight and then blocked with 1% BSA in HEPES buffer (10 mM HEPES, 136 mM NaCl, 2.7 mM KCl, 2 mM MgCl₂, pH 7.45) for 30 minutes. Whole blood from healthy donors was taken into citrate, thrombin inhibited (40 μM PPACK) and recalcified (3.75 mM MgCl₂ and 7.5 mM CaCl₂). Blood was preincubated for 10 minutes with either vehicle (PBS) or 500 nM nanobody 2.21 or 35, prior to perfusion through the Maastricht flow chamber for 3.5 minutes. Two endpoint brightfield images were taken while flowing labeling buffer (HEPES buffer, 2 mM CaCl₂, 1 U/mL heparin, 5.5 mM Glucose, 0.1% BSA and Annexin V AF568 (ThermoFisher)) to assess PS-exposure, AF647-anti-CD62P mAb (for CD62P expression, BioLegend), and anti-fibrinogen FITC Ab (for integrin αIIbβ3 activation, DAKO)). Unbound label was washed off with rinse buffer (HEPES buffer, 2 mM CaCl₂, 1 U/mL heparin, 5.5 mM Glucose and 0.1% BSA) and fluorescence images of three random fields of view were acquired with an EVOS AMF4300 microscope (Life Technologies). Brightfield and fluorescence images were quantified for surface area coverage by specific semi-automated ImageJ scripts {van Geffen et al 2019}. Per donor (n≥3) and treatment 2 replicate runs were performed. For the Horm collagen flow experiments all generated data was averaged between donor and treatment per parameter and tested for statistical significance using an unpaired t-test with Mann Whitney correction and GraphPad Prism 7.00. For the atherosclerotic plaque data, subtraction heatmaps were made using the program R. Raw average values over all donors and per parameter were univariate-normalized at a scale of 0-10. Next, control values were subtracted from the treatment values, differences that were statistically significant (P<0.05) by a one-way ANOVA were then summarized in the heatmaps to only visualize relevant effects.

### Surface Plasmon Resonance

Surface plasmon resonance experiments were performed using a Biacore T200 instrument (GE Healthcare). GPVI was immobilised directly onto the CM5 chip using amine-coupling. Reference surfaces were blocked using 1M ethanolamine pH 8. All sensograms shown are double reference subtracted and at least two replicates were injected per cycle as well as experimental replicates of n=3. Experiments were performed at 25° C. with a flow rate of 30 μL/min in HBS-P running buffer (10 mM HEPES pH 7.4, 0.15 M NaCl, 0.005% v/v surfactant P20). Each concentration of Nb2 was run as follows; 120 sec injection, 900 sec dissociation. Kinetic analysis was performed using the Biacore T200 Evaluation software using a global fitting to a 1:1 binding model.

### Crystallisation and Structure Determination

Crystallisation of the GPVI-Nb2 complex was performed using the GPVI N72Q variant and untagged Nb2. Both proteins were mixed at 75 μM and the complex was purified using a Superdex 200 increase 10/300 GL gel filtration column equilibrated in 20 mM Tris pH 7.4, 140 mM NaCl. The formation of the complex was confirmed by SDS PAGE and spin concentrated to 5 mg/mL. Crystals were generated in 0.2 M calcium acetate, 0.1M sodium cacodylate 6.5, 18% PEG8K and diffraction data was collected at the Diamond Light Source i24 beamline.

The CCP4 software suite was used for structure determination. Molecular replacement was performed in PHASER using 2GI7 and 5TP3 as templates. This was followed by model building in COOT and multiple rounds of refinement in REFMAC. Data collection and refinement statistics are shown in Table 2.

TABLE 2
Crystallographic data collection and refinement statistics.

| Data collection | Value |
|---|---|
| Space group | P 21 21 21 |
| Cell dimensions | |
| a, b, c (Å) | 69.91 84.75 124.04 |
| α, β, γ (°) | 90.00 90.00 90.00 |
| Resolution (Å) | 84.57-2.5 |
| R merge | 0.122 (0.595)* |
| l/σl | 8.67 (2.69)* |
| Completeness (%) | 100 (98.6)* |
| Redundancy | 6.3 (6.4)* |
| Wavelength | 0.96864 Å |
| Refinement | |
| No of reflections | 26173 |
| R_{work}^{b}/R _{free} (%) | 0.176/0.232 |
| No. atoms | |
| Protein | 4664 |
| Ca²⁺ | 1 |
| Water | 271 |
| B-factors (Å²) | |
| Protein | 43.91 |
| Metal | 24.33 |
| Water | 41.78 |
| R.m.s deviations | |
| Bond lengths (Å) | 0.0084 |
| Bond angles (°) | 1.568 |

*Values in parentheses are for highest-resolution shell.
aRmerge = Σh Σi| <Iₕ> − I_{h,i}|Σh Σi I_{h,l} where I is the observed intensity and <Iₕ> is the average intensity of multiple observations from symmetry-related reflections calculated.
bRwork = Sum(h) ||Fo|h − |Fc|h|/Sum(h)|Fo|h, where Fo and Fc are the observed and calculatedstructure factors, respectively. Rfree computed as in Rwork, but only for (5%) randomly selected reflections, which were omitted in refinement, calculated using REFMAC.

Nb35 was also co-crystallised using the same methods as with Nb2. Crystals were grown in 1.6 M magnesium sulphate hydrate, 0.1 M MES monohydrate pH 6.5 using a protein concentration of 6 mg/ml. Maximum resolution for this structure was 3.4 Å. The final model used, although not complete, had an Rwork of 0.31 and an Rfree of 0.41.

### Nuclear Factor of Activated T-Cell (NFAT) Reporter Assay

The Nuclear factor of activated T-cell (NFAT) reporter assay was used for GPVI-signalling detection, following the protocol documented by Tomlinson et al. {Tomlinson, 2007}. DT-40 cells were transfected with 2 μg of full length GPVI, FcR-γ chain and NFAT controlled luciferase reporter construct. Transfected cells were incubated with 100 nM of each nanobody for 15 min followed by stimulation upon the addition of 10 μg/ml collagen. All readouts were expressed as a percentage of the signal from collagen alone. Expression was confirmed by cell labelling samples with HY101 antibody followed by anti-mouse Alexa Fluor-647 secondary antibody staining and performing flow cytometry. The samples were acquired (FL1 and 4) and analysed in an Accuri C6 flow cytometer (BD Biosciences, USA).

### Competitive Binding Assay Details

Competition ELISA experiments were performed by coating Maxisorb ELISA plates with 10 nM GPVI-Fc and binding 100 nM of each nanobody. Un-tagged Nb2 was then added in increasing concentrations and the binding of tagged nanobodies was determined using anti-His HRP.

### Nanobody 2 Stability Tests

Freshly made un-tagged nanobody 2 (4.2 mg/ml in PBS) was stored at 4° C. over a period of 72 hours. Samples were collected at 0, 24, 48 and 72 hours for ELISA and nanodrop analysis. 1 ml of Nb2 was also filtered using a Whatman 0.2 um syringe filter, and 50 μL was snap-frozen and thawed to determine the effects of both syringe filtering and freeze-thawing on nanobody function. The freeze-thawed sample was only tested between 0-48 hours after thawing. ELISA displacement assays were performed on each sample as described in ‘Solid-phase binding assay’. Nb2 concentrations were determined at each time point by measuring absorbance a 280 nm using a nanodrop ND-1000 spectrophotometer.

### Western Blot of Signalling Proteins

Washed platelets were preincubated with either PBS or 500 nM Nb2 or non-inhibitory Nb53, before being stimulated with 10 μg/ml fibrillar Horm collagen or 500 μg/ml pooled plaque homogenate on a shaking plate incubator (Eppendorf). Whole cell lysates were processed as described in Nicolson et al. {Nicolson, 2018} and then investigated for total phospho-tyrosine (4G10, Millipore, 05-321), phospho-PLCγ2 (Y1217, Cell Signalling, 3871S), phospho-LAT (Y200, Abcam, ab68139), phospho-Syk (Y525/526, Cell Signalling, 2710S) as well as total Syk (4D10, St. Cruz, sc1240) as a loading control. Results were visualized on film, as well as imaged for quantification with an Odyssey Fc System (LI-COR Biosciences) in combination with Image studio lite v5.2.

### Labelling of Nb28

Nb28 was Fluorescently Labelled Using N-Hydroxysuccinimide (NHS)-ester Labelling of Free Amino Groups with AlexaFluor 647 Dye According to the Manufacturer's Instructions Using a Dye:Protein (V:V) Ratio of 1:40

### Visualization of GPVI Clustering Under Flow

Blood was prepared for flow as described above. Vehicle or 500 nM Nb2 were preincubated for 10 min then GPVI was labelled by addition of 100 nM of Nb28-AF647 before blood was perfused through the flow chamber at shear rate of 1000/s as above. End point images of GPVI were taken on an EVOS AMF4300 microscope (Life Technologies).

### Statistics

Results are shown±standard deviation. ELISA assays were performed with n=3 whereas NFAT assays were performed with n=5. Student two-tailed t test was used and P<0.05 taken as significant.

### Results

### Generation of Nanobodies Against GPVI

Nanobodies where generated by VIB nanobody core. Immunisation was performed with GPVI-Fc and full DNA sequence of each positive nanobody clone was supplied in pMECS expression vector.

In total 54 nanobodies where obtained. The sequence of each nanobody is disclosed in SEQ ID NOS: 1-54.

### Evaluation of Nanobodies as Blocking Agents

We used a nuclear factor of activated T-cells (NFAT) reporter assay to investigate GPVI signalling in a transfected cell line (FIG. 1a). This assay takes advantage of Src-independent ITAM signalling which results in NFAT-dependent expression of a luciferase reporter {Tomlinson, 2007}. Collagen stimulated an 8.3±2.8 fold increase in NFAT activity. Nbs 2, 21 and 35 inhibited the increase by greater than 80%, and Nbs 1, 5, 19, 22, 25, 33 and 44 inhibited this by more than 40%. Several nanobodies, Nbs 6, 24, 28-30 and 49, increased the response to collagen GPVI and the remainder either had no effect or resulted in inhibition <60%. The marked potency of Nbs 2, 21 and 35 is in line with their high affinity for binding to GPVI (FIG. 1a).

### Testing of Nanobodies on Recombinant GPVI

The recombinant GPVI used for immunisation and testing consisted of the extracellular D1 and D2 domains fused with the Fc domain from IgG. Immunisation with GPVI-Fc yielded 54 distinct nanobody sequences. The Fc domain was used to test whether the nanobodies recognise this region. The 54 nanobodies were categorized into 33 distinct binding classes on the basis of their complementary determining loop (CDR) 3 regions, which is the region that confers ligand specificity. Nanobodies (1 μM) from each binding class were tested for their ability to recognise a recombinant GPVI-Fc coated surface (FIG. 1b). The degree of nanobody binding varied between classes and could be categorised into strong (>50%), moderate (>20%) and weak binders (<20%). Strong binders included Nb2, 21, 35 and 52. Moderate binders included Nb7, 18, 28 and 54. All other nanobodies were considered to be weak binders.

### Binding of Nanobodies to Resting and Activated Platelets

The generation of reagents that bind exclusively to the dimeric form of recombinant GPVI and which recognise an increased number of receptors on stimulated platelets has been interpreted as evidence of receptor dimerization {Jung et al 2012}. Studies were therefore undertaken to compare the binding of the nanobodies to resting platelets and platelets stimulated by CRP. These studies focussed on 33 of the nanobodies from each binding class. This included nanobodies that showed weak binding (FIG. 1) in case they recognise a unique conformation in resting or stimulated platelets.

The binding to platelets was detected by flow cytometry. The nanobodies showed three distinct binding patterns upon platelet activation with CRP and PAR1 (i) 19 nanobodies showed no change compared to resting (type I); (ii) 6 nanobodies showed a small increase in binding (1.2-19.7% and 2.4-15.7% increase for CRP and PAR1 respectively) and (iii) 8 nanobodies showed a small decrease in binding (17.5-44% and 2.0-16.8% decrease for CRP and PAR1 respectively) (FIG. 1bi-iii). These results indicate that none of the nanobodies show a marked difference in recognition of GPVI in resting and stimulated platelets.

### Concentration Response Relationship for Binding of Nbs 2, 21 and 35 to GPVI and Inhibition of Platelet Aggregation

The most potent nanobodies, Nb2, 21 and 35, were further tested in their ability to inhibit GPVI function. Firstly, their binding to platelets was tested using flow cytometry. All three nanobodies exhibited similar binding to resting and activated platelets in the presence of PAR1 activating peptide (200 μM) (FIG. 2) suggesting that they recognise both monomeric and dimeric GPVI. Previously, Jung et al 2012 have reported that thrombin stimulates dimerisation of GPVI. Further studies were performed on these nanobodies to investigate the concentration response relationship in binding to GPVI and for inhibition of platelet aggregation and adhesion under flow. Nbs 2, 21 and 35 inhibited platelet aggregation to collagen (5 μg/mL and CRP (10 μg/ml) with IC50 values of 172, 85 and 115 nM for collagen, and 1, 22 and 1 nM for CRP, respectively (FIG. 3). The differential IC50 values is likely to reflect binding of collagen to a second receptor on platelets, integrin α2β1.

In a solid-phase binding assay, all three nanobodies blocked the binding of GPVI-Fc (100 nM) to a collagen surface with IC50 values for Nbs 2, 21 and 35 of 18, 61 and 39 nM, respectively (FIG. 4a). The binding affinity of the most potent of the three nanobodies, Nb2, to immobilised GPVI and GPVI-Fc was determined by SPR with a calculated equilibrium dissociation constant (K_{D}) of 0.7 nM±0.03 nM and 0.58 nM±0.06 nM for GPVI-Fc and GPVI respectively (FIG. 4b-c). This binding affinity is approximately 25-fold higher than that of the full-length inhibitory GPVI antibody 9O12 {Lebozec et al 2017} The similar binding affinities to monomeric and dimeric GPVI is consistent with the observation that binding of Nb2 to platelets is not altered upon thrombin stimulation. To assess the effect of the most potent anti-GPVI nanobodies on platelet activation and subsequent thrombus formation a whole blood flow adhesion assay was performed. Blood from healthy donors was preincubated with either PBS or 500 nM Nb2, 21 or 35 for 10 minutes, thrombin inhibited, recalcified and then flown over collagen at a shear rate of 1000/s. Quantitative analysis of the images demonstrated that all three Nbs had no significant effect on platelet adhesion under flow (FIG. 5b), but that they inhibited the formation of multilayered aggregates (FIG. 5c). The platelets that did adhere also had abrogated PS-exposure relative to untreated controls (FIG. 5d). The abolition of aggregation and PS exposure, but not adhesion, under flow is consistent with results in GPVI-deficient patients {Nagy et al 2020}.

### Nb2 Binds Close to the CRP Binding Epitope and Reveals a Novel Domain-Swapped Dimeric GPVI Structure

Studies were designed to solve the crystal structure of the most potent of the nanobodies, Nb2, with recombinant monomeric GPVI. For these studies, the single N-glycosylation site at N72 was mutated to glutamine (GPVI NQ). GPVI NQ was mixed with Nb2 in a 1:1 ratio at a concentration of 75 μM and purified as a complex by gel filtration.

The crystal structure of this complex was solved with a resolution of 2.2 Å. The structure revealed a novel GPVI dimer conformation (FIG. 3). The dimer interface consisted of two domain-swapped D2 domains formed through the extension of the C-C′ loop region (labelling in accordance with Horii et al. {Horii, 2006}). This extended loop forms a domain swap hinge that extends outwards and folds with an adjacent D2 (FIG. 6a). This is in stark contrast with the back to back D2 dimer reported in previous crystal structures {Horii, 2006}.

The Nb2 binding site was mapped within D1 adjacent to the CRP binding site. Residues involved in the Nb2-GPVI binding interface is shown in FIG. 6b and below. The primary interaction interface is found towards the top of the CRP binding groove within the D1 C′ β-sheet and 3₁₀ helix found between βE and F strands. This forms a primary binding pocket and polar contacts between nanobody residues within the CDR3 loop with GPVI residues S45-Y47 found within the D1 C′ β-sheet, and S61 found in the short 3₁₀ helix. Additional contacts within the primary binding site are made between Nb2 CDR1 residue Y31 and GPVI residue Q48. A secondary interaction pocket is located away from the CRP binding groove made by residues Q1 and Y115 of Nb2 interacting with E21, P56 and A57 of GPVI. The GPVI binding site lies entirely within the CDR3 loop of Nb2 apart from Q1 and Y31. Sequence alignment of the CDR3 region of the nanobodies identified herein, shows that a number of the nanobodies display significant sequence identity or conservative substitutions, particularly in region around amino acids 99-102 and/or 112-115 (numbering according to the Nb2 nanobody shown below. An alignment of the top 3 inhibitory nanobodies (Nb21, Nb2 and Nb35) shows a significant degree of sequence identity or similarity in the CDR3 region, which is principally involved in binding to GPVI (see below). This information, coupled with the binding data (discussed below), suggests that many of the nanobodies, and in particular Nb2, Nb21 and/or Nb35, may share the same binding region in GPVI. The Nb2 binding site is adjacent to the CRP binding interface (FIG. 4b) with the CDR2 loop extending towards the CRP binding site which would induce steric clashes between both ligands. In addition, the binding of the CDR3 loop to the short C′ β-sheet of D1 the top of the CRP binding groove induces a small shift of approximately 1.5 Å which causes a small distortion of the groove (FIG. 6c). A Ca²⁺ cation can be found bridging two Nb2 subunits through interactions with the side chain carboxyl group of E6 and peptide backbone carbonyl of G119 from two Nb2 subunits and two water molecules resulting in an octahedral arrangement.

The GPVI residues (shown in bold) that make polar contacts with Nb2 are shown below:

Q¹SGPLPKPS L¹⁰QALPSSLVP L²⁰EKPVTLRCQ G³⁰PPGVDLYRL E⁴⁰KLSSSRYQD Q⁵⁰AVLFIPAMK R⁶⁰SLAGRYRCS Y⁷⁰QNGSLWSLP S⁸⁰DQLELVAT

Binding residues=E21, S45, R46, Y47, Q48, P56, A57, S61

Nb2 residues (shown in bold) that make polar contacts with GPVI are shown below:

Q¹VQLQESGG G¹⁰LVQPGGSLR L²⁰SCAAAGFTF D³⁰YYAIAWFRQ A⁴⁰PGKEREGVS C⁵⁰ISSSDGTTY Y⁶⁰ADSVKGRFT I⁷⁰SKDNAKNTM Y⁸⁰LQMNSLKPE D⁹⁰TAVYYCATS P¹⁰⁰LYSTNDRCI S¹¹⁰EDYDYVVGQG T¹²⁰QVTVSSLVPR-

Binding residues=Q1, Y31, S99, P100, Y102, T104, N105, E111, D112, D114, Y115

CDR3 Sequence alignment of the top 3 inhibitory nanobodies

| | | |
|---|---|---|
| nb21 | 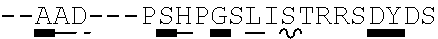 | 20 |
| nb2 | 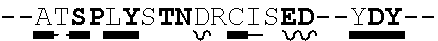 | 19 |
| nb35 | 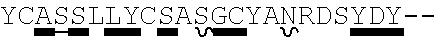 | 23 |
| | *:. : . . ** | |

Amino acids shown in bold for nb2 are residues shown to make polar contacts with GPVI
[symbol] identical amino acid
[symbol] very highly conserved substitution
[symbol] highly conserved substitution (from Blosum90 matrix)
[symbol] conserved substitution (from Blosum65 matrix)

In summary, the crystallisation of the Nb2-GPVI complex has revealed the Nb2 binding site lies in close proximity to the CRP binding site and induces a small conformational change in D1, and we also make the novel observation of a domain swap between the D2 domains.

Competitive binding of nanobody 2 with other nanobodies reveals the majority of nanobodies tested, excluding nanobodies 6, 11, 12, 36 and 53, are displaced by Nb2 when binding to GPVI (FIG. 7a). This suggests the competed nanobodies bind to similar/overlapping binding sites and may share key binding residues on GPVI.

This was further shown by co-crystallising GPVI with Nb35. The diffraction data had a maximum resolution of 3.4 Å. The Nb35 binding site directly overlays with the binding site of Nb2 (FIG. 7b) and is consistent with the competition data. Although the binding site is clear, the current model generated from this dataset is currently not of high enough quality to map the individual binding residues of Nb35. Despite this, the overall positioning for the nanobody binding site on GPVI is accurate.

### Nb2 Stability Tests

Nb2 stability at 4° C. was tested by comparing the ability for nb2 to displace GPVI-Fc binding to a collagen surface after 0-, 24-, 48- and 72-hours storage at 4° C. (FIG. 8). Nb2 was stored in PBS with no added preservatives. In addition, samples of nb2 that were freeze-thawed and an additional sample that had been filtered were also tested. IC50 values were consistent throughout all samples on all days (table 3) suggesting nb2 does not lose activity over 72 hours at 4° C., and freeze-thawing and filtering also have no effect. Concentration readings by nanodrop were also consistent in all samples suggesting no degradation of Nb2 (table 4). There was an observed reduction in protein concentration of approximately 3% when filtering but it is not abnormal for small losses in protein yield when filtering. These results show nb2 can be frozen, filtered and kept in the fridge for at least 72 hours without losing activity.

TABLE 3
Calculated IC50 values for the displacement of
GPVI-Fc binding to collagen by nanobody 2.

| | Day 0 | Day 1 | Day 2 | Day 3 |
|---|---|---|---|---|
| Non-filtered | 37.0 nM | 54.4 nM | 34.0 nM | 32.4 nM |
| Filtered | 45.7 nM | 40.3 nM | 35.2 nM | 37.1 nM |
| Freeze/thawed | 46.5 nM | 29.8 nM | 44.0 nM | N/A |

TABLE 4
Calculated Nb2 concentrations by measuring absorbance at 280 nm.

| | Day 0 | Day 1 | Day 2 | Day 3 |
|---|---|---|---|---|
| Non-filtered | 4.20 ± 0.01 mg/ml | 4.25 ± 0.01 mg/ml | 4.21 ± 0.01 mg/ml | 4.22 ± 0.02 mg/ml |
| Filtered | 4.07 ± 0.02 mg/ml | 4.08 ± 0.01 mg/ml | 4.09 ± 0.02 mg/ml | 4.11 ± 0.01 mg/ml |
| Freeze/thawed | 4.2 ± 0.01 mg/ml | 4.2 ± 0.02 mg/ml | 4.23 ± 0.02 mg/ml | 4.25 ± 0.03 mg/ml |

### Tetravalent Nb2 (Nb2-4) Activates Platelets

Functional studies were undertaken on washed platelets. Divalent Nb2, Nb2-,2, is unable to activate platelets and blocks aggregation by collagen and CRP with a 2-5 fold greater potency that monovalent Nb2 (FIG. 9). The increase in affinity is explained by binding to two GPVI receptors and therefore is the net sum of affinity and avidity. In contrast, low nanomolar concentrations of tetravalent Nb2 (Nb2-4) stimulates powerful aggregation of platelets which is blocked by a high concentrations of monovalent Nb2 and Fab of the GPVI monoclonal antibody, JAQ1 (FIG. 9). Aggregation is also blocked by inhibitors of Src and Syk tyrosine kinases consistent with activation of GPVI. These results demonstrate that Nb2-4 is a novel agonist at GPVI.

### Nb2, Nb21 and Nb35 Effectively Block Thrombus Formation and Platelet Activation Under Flow Over Atherosclerotic Plaque.

Pooled atherosclerotic plaque represents a physiological ligand for platelets that is rich in collagen. The ability of Nb2, Nb21 and Nb35 to inhibit plaque-induced platelet activation and thrombus formation under flow was tested in a flow adhesion assay (FIG. 10). In contrast to flow over purified collagen I (FIG. 5), all three Nbs significantly inhibited platelet adhesion to plaque under flow. They also abolished thrombus formation and inhibited the platelet activation markers of p-selectin expression, integrin activation and PS-exposure, shown by the lighter grey squares in the subtraction heatmaps (FIG. 10b). This demonstrates that the three Nbs effectively inhibit platelet activation and thrombus initiation and formation triggered by plaque.

### Nb2 Strongly Inhibits Plaque-Mediated GPVI Signaling

Western blot was used to investigate the effect of Nb2 on GPVI signalling by examining downstream phosphorylation following stimulation with plaque or collagen I. Both collagen I and plaque induced strong tyrosine phosphorylation in platelets (FIG. 11a). Preincubation of platelets with Nb2 caused a visible reduction in global platelet tyrosine phosphorylation to plaque stimulation, and strongly inhibited phosphorylation of GPVI downstream signalling proteins Syk Y525/526, LAT Y200 and PLCγ2 Y1217 in both plaque and collagen I stimulated platelets (FIG. 11a). Non-inhibitory Nb53 had no effect. Quantitation (FIG. 11b) revealed strong and consistent reduction of phosphorylation by Nb2 in response to plaque, whereas a weaker and more variable response was observed to collagen I, probably reflecting the involvement of other receptors in platelet binding to purified collagen I.

### Non-Inhibitory Nb28 Does Not Compete With Nb2 for GPVI Binding and Can Be Used for Imaging of GPVI

In order to visualise the localisation of GPVI in platelets we required a non-inhibitory anti-GPVI Nb that could be used in imaging studies. Nb28 strongly binds GPVI but does not inhibit GPVI-collagen interactions (FIG. 1). To further test the validity of using Nb28 in imaging studies we verified that it did not inhibit platelet aggregation in response to collagen (FIG. 12a) or thrombus formation under flow on collagen I (FIG. 12b). We then fluorescently labelled Nb28 with AlexaFluor647 (Nb28-AF647) and assessed binding to platelets, as well as competitive binding between inhibitory Nb2 and Nb28-AF647 using flow cytometry (FIG. 12c, d). No significant difference was observed between platelets labelled with Nb28-AF647 alone or those pre-incubated with unlabelled Nb2 (FIG. 12d). These results indicate that Nb28 is suitable for imaging GPVI under flow conditions as it does not interfere with thrombus formation. In addition, Nb28 binds to a different epitope in GPVI, which is not blocked by prior Nb2 binding to GPVI, making Nb28-AF647 suitable for imaging of GPVI in experiments where Nb2 is also present.

### Nb28-AF647 Permits the Visualisation of the Disruptive Effect of Nb2 on GPVI Clustering

To investigate the distribution of GPVI on platelets adhering and forming thrombi under flow, we pre-incubated whole blood with 100 nM Nb28-AF647, before perfusing collagen I microspots and taking fluorescent images. On platelets adhered and forming thrombi on collagen I, bright long clusters of GPVI were frequent and corresponded to the large collagen fibres visible in the brightfield images (arrows; FIG. 13a). To investigate the effect of Nb2 on GPVI localisation we preincubated blood with Nb2 and Nb28-AF647 before flow. Pre-treatment with Nb2 disrupted the GPVI localization to the fibres and no organized clusters were seen along the collagen (arrows). These results show that fluorescently labelled Nb28 is a useful tool for visualising GPVI in platelets and can be used in whole blood flow assays. Also, Nb2 effectively disrupts GPVI clustering on collagen, providing evidence that clustering may be important for thrombus formation under flow.

### Discussion

In this study we have developed a range of nanobodies to dimeric GPVI and characterised these on a variety of assays, and mapped the binding site of the most potent, Nb2, to GPVI. These studies show the following: (i) a range of nanobodies that bind to GPVI with inhibitory and non-inhibitory properties; (ii) none of the nanobodies bind preferentially to resting or stimulated platelets; (iii) the most potent of these, Nb2, 21 and 35, bind with nanomolar affinity and block collagen-induced NFAT activation, platelet aggregation and thrombus formation over collagen and atherosclerotic plaque material; (iv) Both Nb2 and Nb35 binds to a site on GPVI which is adjacent to the collagen binding site; (v) Nb2 forms a complex with a novel domain configuration of GPVI; and vi) Fluorescently tagged non-blocking Nb28 can be used to label GPVI for localisation experiments. Together, the nanobodies form a library of agents for probing GPVI function in platelets.

The crystal structure of Nb2 bound GPVI reveals Nb2 interacts with the top of the CRP binding groove and, although not directly overlapping with the CRP binding site, is close enough to sterically hinder the binding to collagen. Inhibition of collagen binding by Nb2 is likely a combination of steric clashes between closely positioned binding sites as well as the distortion of the CRP binding groove shown in FIG. 4c indicative of a mixed mode allosteric and competitive inhibitor. Nb2 provides new scaffolds and agents that can be used as anti-thrombotic drugs to treat cardiovascular disease.

### REFERENCES

1. Nieswandt B, Watson S P. Platelet-collagen interaction: is GPVI the central receptor? Blood. 2003; 102(2): 449-461.
2. Mammadova-Bach E, Ollivier V, Loyau S, et al. Platelet glycoprotein VI binds to polymerized fibrin and promotes thrombin generation. Blood. 2015; 126(5):683-691.
3. Moroi M, Jung S M. Platelet glycoprotein VI: its structure and function. Thromb Res. 2004; 114(4):221-233.
4. Zheng Y M, Liu C, Chen H, Locke D, Ryan J C, Kahn M L. Expression of the platelet receptor GPVI confers signaling via the Fc receptor gamma -chain in response to the snake venom convulxin but not to collagen. J Biol Chem. 2001; 276(16):12999-13006.
5. Lecut C, Arocas V, Ulrichts H, et al. Identification of residues within human glycoprotein VI involved in the binding to collagen: evidence for the existence of distinct binding sites. J Biol Chem. 2004; 279(50):52293-52299.
6. Ezumi Y, Shindoh K, Tsuji M, Takayama H. Physical and functional association of the Src family kinases Fyn and Lyn with the collagen receptor glycoprotein VI-Fc receptor gamma chain complex on human platelets. J Exp Med. 1998; 188(2):267-276.
7. Watson S P, Auger J M, McCarty O J, Pearce A C. GPVI and integrin alphaIIb beta3 signaling in platelets. J Thromb Haemost. 2005; 3(8):1752-1762.
8. Jung S M, Moroi M, Soejima K, et al. Constitutive dimerization of glycoprotein VI (GPVI) in resting platelets is essential for binding to collagen and activation in flowing blood. J Biol Chem. 2012; 287(35):30000-30013.
9. Miura Y, Takahashi T, Jung S M, Moroi M. Analysis of the interaction of platelet collagen receptor glycoprotein VI (GPVI) with collagen. A dimeric form of GPVI, but not the monomeric form, shows affinity to fibrous collagen. J Biol Chem. 2002; 277(48):46197-46204.
10. Poulter N S, Pollitt A Y, Owen D M, et al. Clustering of glycoprotein VI (GPVI) dimers upon adhesion to collagen as a mechanism to regulate GPVI signaling in platelets. J Thromb Haemost. 2017; 15(3): 549-564.
11. Onselaer M B, Hardy A T, Wilson C, et al. Fibrin and D-dimer bind to monomeric GPVI. Blood Adv. 2017; 1(19): 1495-1504.
12. Raynal N, Hamaia S W, Siljander P R, et al. Use of synthetic peptides to locate novel integrin alpha2beta1-binding motifs in human collagen III. J Biol Chem. 2006; 281(7):3821-3831.
13. Ungerer M, Rosport K, Bültmann A, et al. Novel antiplatelet drug revacept (Dimeric Glycoprotein VI-Fc) specifically and efficiently inhibited collagen-induced platelet aggregation without affecting general hemostasis in humans. Circulation. 2011; 123(17):1891-1899.
14. Nagy M, Perrella G, Dalby A, et al. Flow studies on human GPVI-deficient blood under coagulating and noncoagulating conditions. Blood Adv. 2020; 4(13):2953-2961.
15. van Geffen J P, Brouns S L N, Batista J, et al. High-throughput elucidation of thrombus formation reveals sources of platelet function variability. Haematologica. 2019;104(6):1256-1267.
16. Tomlinson M G, Calaminus S D, Berlanga O, et al. Collagen promotes sustained glycoprotein VI signaling in platelets and cell lines. J Thromb Haemost. 2007; 5(11):2274-2283.
17. Bannas P, Hambach J, Koch-Nolte F. Nanobodies and Nanobody-Based Human Heavy Chain Antibodies As Antitumor Therapeutics. Front Immunol. 2017; 8:1603.
18. Lebozec K, Jandrot-Perrus M, Avenard G, Favre-Bulle O, Billiald P. Design, development and characterization of ACT017, a humanized Fab that blocks platelet's glycoprotein VI function without causing bleeding risks. MAbs. 2017; 9(6):945-958.
19. Horii K, Kahn M L, Herr A B. Structural basis for platelet collagen responses by the immune-type receptor glycoprotein VI. Blood. 2006; 108(3):936-942.
20. Vinke C, Loris R, Saerens D, Martinz-Rodriguez S, Muyldermans S, Conrath K, General strategy to humanize a camelid single-domain antibody and identification of a universal humanized nanobody scaffold. J Biol Chem, 2009.
21 Elvin A. Kabat, Tai Te Wu, Harold M. Perry, Kay S. Gottesman, Carl Foeller Sequence of proteins of immunological interest, U.S. department of health and human service, 1991.
22. Needleman, Saul B. & Wunsch, Christian D. (1970). “A general method applicable to the search for similarities in the amino acid sequence of two proteins”. Journal of Molecular Biology. 48 (3): 443-53.

1. A nanobody, or an antigen binding fragment thereof, which is capable of specifically binding GPVI.

2. The nanobody, or an antigen binding fragment thereof according to claim 1 with a Kd value of less than 500 nM, 250 nM, 100 nM, 50 nM, 25 nM, 10 nM, 5 nM, or 1 nM.

3. The nanobody, or an antigen binding fragment thereof according to claims 1 which does not bind to one or more (such as 2, 3 ,4, 5 or 6) of the following GPVI residues: R38, E40, R67, Q71, W76.

4. The nanobody, or an antigen binding fragment thereof according to claim 1 which binds to one or more or more of the following residues in human GPVI: E21, S45, R46, Y47, Q48, P56, A57 and S61.

5. The nanobody, or an antigen binding fragment thereof according to claim 4, which binds to one or more or more of the following residues in human GPVI: S45, R46, Y47, Q48 and/or S61.

6. The nanobody, or an antigen binding fragment thereof according to claim 1 which comprises at least 1, 2, 3, 4, 5, 6 or more of the following residues, or a conservative substitution thereof: S99, P100, Y102, T104, N105, E111, D112, D114, Y115.

7. The nanobody, or an antigen binding fragment thereof according to claim 1 which is at least 85%, 90%, 95%, 98%, 99%, or 100% identical with SEQ ID NOS: 1-54; SEQ ID NOS: 1, 2, 5, 18, 21, 22, 25, 33, 35, 44; SEQ ID NOS: 2, 21, 22, 25, 35; or SEQ ID NOS: 2, 21 and 35.

8. The nanobody, or an antigen binding fragment thereof according to claim 1 comprising at least the CDR3 sequence, optionally further comprising the CDR1 and/or CDR3 sequences highlighted in Table 1.

9. A nanobody, or an antigen binding fragment thereof that cross-competes with and binds to the same epitope on GPVI as a nanobody or antigen fragment thereof according to claim 1.

10. A conjugate or fusion protein comprising a nanobody, or an antigen binding fragment thereof according to claim 1 and another moiety.

11. The conjugate or fusion protein according to claim 10, wherein the other moiety is a half-life extending moiety.

12. The conjugate or fusion protein according to claim 11, wherein the half-life extending moiety is an Fc domain, a multimerization domain, human serum albumin (HSA), or bovine serum albumin (BSA).

13. The conjugate or fusion protein according to claim 10, wherein the other moiety is a therapeutic drugs, toxin, cytokine, radionuclide, enzyme, or diagnostic molecule or tag.

14. A polynucleotide molecule encoding a nanobody or antigen binding fragment according to claim 1.

15. A pharmaceutical formulation comprising a nanobody or antigen binding fragment according to claim 1, optionally together with a pharmaceutically acceptable excipient therefore.

16. A method of treating or preventing diseases arising from processes of blood platelet aggregation, as well as other related conditions or thrombosis, said method comprising administering to a subject in need thereof a nanobody or antigen binding fragment thereof of claim 1.

17. (canceled)

18. The method of claim 16, wherein the disease or condition is acute and chronic vascular diseases associated with intraarterial and/or intravenous thrombosis, such as acute coronary syndrome/acute myocardial infarction, chronic coronary syndrome/stable coronary artery disease, transient ischaemic attack, stroke, peripheral vascular disease, deep vein thrombosis, thromboprophylaxis in patients with atrial fibrillation, thromboprophylaxis in patients with recent medical or surgical admission to hospital, pulmonary embolism, sepsis-related coagulopathy, COVID-19 associated thrombosis, thrombosis related to cardiac devices (eg. left ventricular assist devices, extra extracorporeal membrane oxygenation and mechanical heart valves), left ventricular thrombosis, thromboprophylaxis in heart failure, thromboprophylaxis in thrombophilic conditions (eg. antiphospholipid syndrome, factor V Leiden) and other forms of inflammation, infection and cancer-driven thrombosis.

19. The method of claim 16, wherein the nanobody or antigen binding fragment thereof is administered in combination with another therapeutic agent such as an antiplatelet drug, including aspirin (and other cyclooxygenase inhibitors), P2Y12 receptor antagonist and GPIIb/IIIa inhibitor, as well as anticoagulants including heparin, warfarin and direct inhibitors of Factor Xa and thrombin, and thrombolytic therapy such as reteplase, alteplase and streptokinase.

20. A method of imaging GPVI, said method comprising the use of a nanobody according to claim 1 to bind and visualise GPVI.

21. The method of claim 20, wherein the nanobody does not compete with GPVI for binding to collagen.

22. The method of claim 21, wherein the nanobody comprises the sequence according to SEQ ID NO:28, optionally encoded by SEQ ID NO: 82.


SEQUENCE LISTING (108 sequences)

SEQ ID NO: 1 - protein, 145 aa, Artificial Sequence
  Synthetic Peptide
QVQLQESGGGLVQAGGSLRLSCASSGFTFSGYQLAWFRHVAPGKEREFVAAIRWSGGITWYADSVKGRFTISRDNAKNTMYLQMNSLKPEETAVYYCAASPLYSNNYLLPDNYDYWGQGTQVTVSSAAAYPYDVPDYGSHHHHHH

SEQ ID NO: 2 - protein, 145 aa, Artificial Sequence
  Synthetic Peptide
QVQLQESGGGLVQPGGSLRLSCAAAGFTFDYYAIAWFRQAPGKEREGVSCISSSDGTTYYADSVKGRFTISKDNAKNTMYLQMNSLKPEDTAVYYCATSPLYSTNDRCISEDYDYWGQGTQVTVSSAAAYPYDVPDYGSHHHHHH

SEQ ID NO: 3 - protein, 146 aa, Artificial Sequence
  Synthetic Peptide
QVQLQESGGGLVQPGGSVRLSCAASGFTLDRFTIGWFRQVPGKEREGVSCISEVAGSIYYADSVKGRFTISRDNAKNTVYLQMNSLKPEDTAVYTCAADPLLYRGGGCLIPHEYPYWGQGTQVTVSSAAAYPYDVPDYGSHHHHHH

SEQ ID NO: 4 - protein, 139 aa, Artificial Sequence
  Synthetic Peptide
QVQLQESGGGLVQPGGSLRLSCAASGFTFSNYAMTWVRQAPGKVVEWVSRIHSGDNSITYAEFVKGRFTISRDNARNTLYLQMNSLKPEDTAVYYCARVVPGFQYRYDYWGQGTQVTVSSAAAYPYDVPDYGSHHHHHH

SEQ ID NO: 5 - protein, 146 aa, Artificial Sequence
  Synthetic Peptide
QVQLQESGGGLVQPGGSLRLSCAASGFTLDSHTIGWFRQAPGKEREGVSGINSSDGSTYYADSVKGRFTISRDNAKNTVYLQMDSLKVEDTAVYYCATTPFSLRGPTWVTPDEFDYWGQGTQVTVSSAAAYPYDVPDYGSHHHHHH

SEQ ID NO: 6 - protein, 143 aa, Artificial Sequence
  Synthetic Peptide
QVQLQESGGGLVQPGGSLRLSCEASGRTFSSYTVGWFRQASGKEREFVGTVNWSGQITTYADSVKGRFTISRQYAENTVYLEMNSLKNEDTAVYYCAAHQGLGTPRTPKQYDYWGRGTQVTVSSAAAYPYDVPDYGSHHHHHH

SEQ ID NO: 7 - protein, 147 aa, Artificial Sequence
  Synthetic Peptide
QVQLQESGGGVVQDGGSLRLSCAASGRTFSSYAMGWFRQAPGKEREFVAAISWSGDSTYSADSVRGRFTISRDNAKNTVYLQMNSLKPEDTAVYYCAADGRNYGLGPLETDSYEYDYWGQGTQVTVSSAAAYPYDVPDYGSHHHHHH

SEQ ID NO: 8 - protein, 146 aa, Artificial Sequence
  Synthetic Peptide
QVQLQESGGGLVQPGGSLRLSCAASGLSLGYYAIGWFRQAPGKEREVVSCDSVADGSTYYADSVLGRFTISRDNPKNTMYLQMNSLKPEDTAVYYCAALFDSGLGDFPPPVEDYDYWGQGTQVTVSSAAAYPYDVPDYGSHHHHHH

SEQ ID NO: 9 - protein, 145 aa, Artificial Sequence
  Synthetic Peptide
QVQLQESGGGLVQAGGSLRLSCAASGFTFDNYAIGWFRQAPGKEREGVSCIGSSDGSTYYADSVKGRFTISRDNAKNTKYLQMNSLKPEDTAVYYCAASPLFVAGICLSEYDYDYWGQGTQVTVSSAAAYPYDVPDYGSHHHHHH

SEQ ID NO: 10 - protein, 143 aa, Artificial Sequence
  Synthetic Peptide
QVQLQESGGGLVQAGGSLRLSCAASGRTFSSSTMGWFRQAPGKEREFVATVNWNGQITTYADSVKGRFTISRQYAENTVYLDMNSLKNEDTAVYYCAAHQGLGTPRTPKQYAYWGRGTQVTVSSAAAYPYDVPDYGSHHHHHH

SEQ ID NO: 11 - protein, 147 aa, Artificial Sequence
  Synthetic Peptide
QVQLQESGGGVVQTGGSLRLSCVASGFSFEDYGIGWFRQAPGKEREGVSYIKNTDGDTTYADSVKGRFTISSDYAKNTVYLQMNDLEPQDAAVYYCAARPPPLNYYSPYLTPREFSYWGQGTQVTVSSAAAYPYDVPDYGSHHHHHH

SEQ ID NO: 12 - protein, 133 aa, Artificial Sequence
  Synthetic Peptide
QVQLQESGGGLVQPGGSLRLSCAASGFTFSSYAMSWVRQAPGKGLEWVATIGGGGAASNVADSVKGRFTISRDNAKNTLYLQMNSLKPDDTAVYYCEESRRGTRGQGTQVTVSSAAAYPYDVPDYGSHHHHHH

SEQ ID NO: 13 - protein, 147 aa, Artificial Sequence
  Synthetic Peptide
QVQLQESGGGLVQTGGSLRLSCAASGRTFSSYAMGWFRQAPGKEREFVAAISWSGDSTYSADSVRGRFTISRDNAKNTVYLQMNSLKPEDTAVYYCAADGRNYGLGPLETDSYEYDYWGQGTQVTVSSAAAYPYDVPDYGSHHHHHH

SEQ ID NO: 14 - protein, 142 aa, Artificial Sequence
  Synthetic Peptide
QVQLQESGGGLVQPGGSLRLSCAASGFTLDNYAIGWFRQAPGKEREGVSCISSSDGSAYYADSVKGRFTISRDNAKNTVNLQMNSLKPEDTAVYYCAADPFWGRCQASDYAYWGQGTQVTVSSAAAYPYDVPDYGSHHHHHH

SEQ ID NO: 15 - protein, 146 aa, Artificial Sequence
  Synthetic Peptide
QVQLQESGGGLVQAGGSLRLSCAPSGRTFSSDAMGWFRQVPGKEREFVAAISWSGGSTYYADSVKGRFTISRDNAKNTVYLQMDSLKPEDTAVYYCAASLLRSYYSPDHTSYEYDYWGQGTQVTVSSAAAYPYDVPDYGSHHHHHH

SEQ ID NO: 16 - protein, 147 aa, Artificial Sequence
  Synthetic Peptide
QVQLQESGGGLVQAGGSLRLSCVASESTFSPYGMGWFRQAPGKEREFVAAISWSGDSTYSADSVRGRFTISRDNAKNTVYLQMNSLKPEDTAVYYCAADGRNYGLGPLETDSYEYDYWGQGTQVTVSSAAAYPYDVPDYGSHHHHHH

SEQ ID NO: 17 - protein, 143 aa, Artificial Sequence
  Synthetic Peptide
QVQLQESGGGLVQAGDSLRLTCAQSRRTFSPYAMAWFRQAPGKEREFVAAVTWSGGSTYYADSVKGRFTVSRDNAKSTLYLQMNSLKPEDTAVYYCAASLFVIAGTPNDEYDYWGQGTQVTVSSAAAYPYDVPDYGSHHHHHH

SEQ ID NO: 18 - protein, 145 aa, Artificial Sequence
  Synthetic Peptide
QVQLQESGGGLVQAGGSLRLSCAASGFTFDDYAIGWFRQAPGKEREGVSCISSSDGSTYYADSVKGRFTIARDNAKNTVYLQMNSLKPEETAVYYCAASPLYYSGSYCRETEYDYWGQGTQVTVSSAAAYPYDVPDYGSHHHHHH

SEQ ID NO: 19 - protein, 146 aa, Artificial Sequence
  Synthetic Peptide
QVQLQESGGGLVQAGGSLRLSCAASGRTFSSDAMGWFRQAPGKEREFVAAIRWSGGSTYYADSVKGRFTISRGNAKNTVYLQMNSLKPEDTAVYYCASSLLNSYYPPDHTSYEYDYWGQGTQVTVSSAAAYPYDVPDYGSHHHHHH

SEQ ID NO: 20 - protein, 147 aa, Artificial Sequence
  Synthetic Peptide
QVQLQESGGGLVQAGGSLRLSCAASGRSSSSYAIAWFRQAPGKEREFAAAISWSGDSTYSADSVRGRFTISRDNAKNTVYLQMNSLKPEDTAVYYCAADGRNYGLGPLETDSYEYDYWGQGTQVTVSSAAAYPYDVPDYGSHHHHHH

SEQ ID NO: 21 - protein, 146 aa, Artificial Sequence
  Synthetic Peptide
QVQLQESGGGLVQPGGSLRLSCAASGRTFTRSIMGWFHQAPGKEREFLAGISWSGANTYYADSVRGRFTISRDNAKNTVSLQMNSLNPEDTAVYYCAADPSHPGSLISTRRSDYDSWGRGTQVTVSSAAAYPYDVPDYGSHHHHHH

SEQ ID NO: 22 - protein, 148 aa, Artificial Sequence
  Synthetic Peptide
QVQLQESGGGLVQAGGSLRLSCAPSGRTISSVDVGWFRQAPGKEREFVAHVTWSGGSTYAADSVKGRFTVSRDNAKNTVYLQMNSLKPEDTAVYYCAAALNGAFRDSWYPALWDEYDYWGQGTQVTVSSAAAYPYDVPDYGSHHHHHH

SEQ ID NO: 23 - protein, 143 aa, Artificial Sequence
  Synthetic Peptide
QVQLQESGGGLVQAGGSLRLSCEASGRTAGSSTIAWFRQAPGKEREFVTTVNWSGQITTYADSVKGRFTISRQYAENTVYLEMNSLKNEDTAVYYCAAHQGLGTPRTPKQYDYWGRGTQVTVSSAAAYPYDVPDYGSHHHHHH

SEQ ID NO: 24 - protein, 147 aa, Artificial Sequence
  Synthetic Peptide
QVQLQESGGGSVQAGDSLRLSCAASGRTFSSYAMAWFRQAPGKEREFVAGIHWSGTITYYADSVKGRFALSRDNAKNTVYLEMNSLKPEDTAVYYCAANNYHCSTYGCTTSPARYDYWGQGTQVTVSSAAAYPYDVPDYGSHHHHHH

SEQ ID NO: 25 - protein, 145 aa, Artificial Sequence
  Synthetic Peptide
QVQLQESGGGLVQPGGSLRLSCAASGFTLDNYAIGWFRQAPGKEREGVSCISSSDGRTYYADSVKGRFTISRDNAKNTMYLQMNSLKPEDTAVYYCATSPLYSDSDRCIWEEYDYWGQGTQVTVSSAAAYPYDVPDYGSHHHHHH

SEQ ID NO: 26 - protein, 143 aa, Artificial Sequence
  Synthetic Peptide
QVQLQESGGGLVQAGGSLRLSCAASGRTFGSYAVGWFRQASGKEREFVGTVNWSGQITTYADSVKGRFTISRQYAENTVYLEMNSLKNEDTAVYYCAAHQGLGTPRTPKQYDYWGRGTQVTVSSAAAYPYDVPDYGSHHHHHH

SEQ ID NO: 27 - protein, 147 aa, Artificial Sequence
  Synthetic Peptide
QVQLQESGGGLVQTGGSLRLSCTASGRAFSRYAMGWFRQAPGKEREFVAAISWSGDSTYSADSVRGRFTISRDNAKNTVYLQMNSLKPEDTAVYYCAADGRNYGLGPLETDSYEYDYWGQGTQVTVSSAAAYPYDVPDYGSHHHHHH

SEQ ID NO: 28 - protein, 139 aa, Artificial Sequence
  Synthetic Peptide
QVQLQESGGGLVQAGDSLRLSCAASGTYSMGWFRQAPGKEREFVAAISWSGGRTNYADSVKGRFTISRDNAKNTVYLQMNSLKPEDTAVYYCSAAIYDNVSSRIGSYTYWGQGTQVTVSSAAAYPYDVPDYGSHHHHHH

SEQ ID NO: 29 - protein, 144 aa, Artificial Sequence
  Synthetic Peptide
  misc_feature (36)..(36): Xaa can be any naturally occurring amino acid
QVQLQESGGGLVQVGGSLRLSCAASGFIFDDYAIGXFRQAPGKEREGVSCISSSDGSTYYADSVKGRFTISRDNAKNTVYLQMNSLKPEDTAVYYCAAGVMSSWRDYKHYGMDYWGKGTQVTVSSAAAYPYDVPDYGSHHHHHH

SEQ ID NO: 30 - protein, 144 aa, Artificial Sequence
  Synthetic Peptide
QVQLQESGGGFVQAGGSLRLSCVASGRTFSTYDMGWFRQAPGKEREFVASILIGGGTYYADSVKGRFTISRDNAKNTVYLQMNSLKPEDTAVYYCAYRAPSSGAYYSNQRAYDYWGQGTQVTVSSAAAYPYDVPDYGSHHHHHH

SEQ ID NO: 31 - protein, 147 aa, Artificial Sequence
  Synthetic Peptide
QVQLQESGGGLVQAGDSLRLSCAASGRTFSNYAMGWFRQEPGKEREFVAGINWSGDSTYYADSVKGRFTISRDNAKNTLYLQMNSLKPEDTAVYYCAAGLFHGSWSERPEAISEYDYWGQGTQVTVSSAAAYPYDVPDYGSHHHHHH

SEQ ID NO: 32 - protein, 146 aa, Artificial Sequence
  Synthetic Peptide
QVQLQESGGGLTQAGGSLRLSCAASGRTFTRSIMGWFRQAPGKEREFLAAITWSGGNTYYADSVRGRFTISRDNAKNTVSLQMNSLKPEDTAVYYCAADPSHLGSLISTRRSDYDSWGRGTQVTVSSAAAYPYDVPDYGSHHHHHH

SEQ ID NO: 33 - protein, 148 aa, Artificial Sequence
  Synthetic Peptide
QVQLQESGGGLVQAGDSLRLSCAASGRTFSSYVMGWFRQAPGKEREFVAAISWSGGSTYYADSVKGRFTISRDNAKNTVYLQMNSLKPEDTAVYYCAAADPPSFYSDYDWPRDHEYDYWGQGTQVTVSSAAAYPYDVPDYGSHHHHHH

SEQ ID NO: 34 - protein, 146 aa, Artificial Sequence
  Synthetic Peptide
QVQLQESGGGLVQAGGSLRLSCAPSGRTFSSDAMGWFRQVPGKEREFVAAISWSGGSTYYADSVKGRFTISRDNAKNTVYLQMDSLKPEDTAVYYCAASLLRSYYPPDHTSYEYDYWGQGTQVTVSSAAAYPYDVPDYGSHHHHHH

SEQ ID NO: 35 - protein, 148 aa, Artificial Sequence
  Synthetic Peptide
QVQLQESGGGLVQAGGSLRLSCAASGVTFDSAAMAWFRQVPGKEREFVAVISTESGGRTDHADSVKGRFLISRDNARHMVYLQMNSLNPEDTAVYYCASSLLYCSASGCYANRDSYDYWGQGTQVTVSSAAAYPYDVPDYGSHHHHHH

SEQ ID NO: 36 - protein, 143 aa, Artificial Sequence
  Synthetic Peptide
QVQLQESGGGLVQAGGSLRLSCEASGRTFSSSTMGWFRQAPGKEREFVATVNWNGQITTYADSVKGRFTISRQYAENTVYLDMNSLKNEDTAVYYCAAHQGLGTPRTPKQYAYWGRGTQVTVSSAAAYPYDVPDYGSHHHHHH

SEQ ID NO: 37 - protein, 145 aa, Artificial Sequence
  Synthetic Peptide
QVQLQESGGGLVQAGGSLRLSCAASGFTFDDYAIGWFRQAPGREREGVSCISDSDGSTYYADSVKGRFTISRDNAKNTMYLQMNSLKPEDTAVYYCAASPLFVAGICLSKYEYDYWGQGTQVTVSSAAAYPYDVPDYGSHHHHHH

SEQ ID NO: 38 - protein, 144 aa, Artificial Sequence
  Synthetic Peptide
QVQLQESGGGLVQTGDSLRLSCATSGGTFSGYELAWFRQAPGKEREFVAHITRSGRATYYLDSVKGRFTISRDNARNTVYLQMNSLKPEDTAVYYCAASLLINPVGVGQNEYDYWGQGTQVTVSSAAAYPYDVPDYGSHHHHHH

SEQ ID NO: 39 - protein, 143 aa, Artificial Sequence
  Synthetic Peptide
QVQLQESGGGLVQAGGSLRLSCAASGLTFGNCDLAWFRQAPGKERETVAAVRRSDGATYYADSVKGRFTISRDNAKNTVYLQMNSLKPEDTAVYYCAASLLTSATIIQPEYDYWGQGTQVTVSSAAAYPYDVPDYGSHHHHHH

SEQ ID NO: 40 - protein, 147 aa, Artificial Sequence
  Synthetic Peptide
QVQLQESGGGLVQAGGSLRLSCAASGRTFSTYSMGWFRQAPGKEREFVAAISWSGDSTYSADSVRGRFTISRDNAKNTVYLQMNSLKPEDTAVYYCAADGRNYGLGPLETDSYEYDYWGQGTQVTVSSAAAYPYDVPDYGSHHHHHH

SEQ ID NO: 41 - protein, 140 aa, Artificial Sequence
  Synthetic Peptide
  misc_feature (13)..(13): Xaa can be any naturally occurring amino acid
  misc_feature (75)..(75): Xaa can be any naturally occurring amino acid
QVQLQESGGGLVXPGGSLRLSCAASTSIFSFDFMAWYRQAPGEQRDLVAALTSNGATNYADSVKGRFTISRDNAXNNVYLQMDSLKPEDTAVYYCNAALRTDSWDRREDYWGQGTQVTVSSAAAYPYDVPDYGSHHHHHH

SEQ ID NO: 42 - protein, 145 aa, Artificial Sequence
  Synthetic Peptide
QVQLQESGGGLVQPGGSLRLSCAASGFTLDYYAIGWFRQAPGKEREGVSCISSNDGSTYYADSVKGRFTISRDNAKNTVYLQMNSLKPEDTAVYYCATSPLYYSGSYCRADEYDYWGQGTQITVSSAAAYPYDVPDYGSHHHHHH

SEQ ID NO: 43 - protein, 143 aa, Artificial Sequence
  Synthetic Peptide
QVQLQESGGGLVQAGDSLRLSCEASGRTFSSYTVGWFRQASGKEREFVGTVNWSGQITTYADSVKGRFTISRQYAENTVYLEMNSLKNEDTAVYYCAAHQGLGTPRTPKQYDYWGRGTQVTVSSAAAYPYDVPDYGSHHHHHH

SEQ ID NO: 44 - protein, 144 aa, Artificial Sequence
  Synthetic Peptide
QVQLQESGGGLVQPGGALRLSCAASGFTLDYYAIGWFRQAPGKEREAVSCISSSDGSTYYADSVKGRFTISRDNAKNTVYLQMNSLKPEDTAVYYCATDPFYSDINCKSYEYAYWGQGTQVTVSSAAAYPYDVPDYGSHHHHHH

SEQ ID NO: 45 - protein, 144 aa, Artificial Sequence
  Synthetic Peptide
QVQLQESGGGLVQTGDSLRLSCATSGGTFSSYEMAWFRQAPGKEREFVAHITRSGRSAYYLDSVKGRFTISRDNARNTVYLQMNSLKPEDTAVYYCAASLLINPVGVGQNEYDYWGQGTQVTVSSAAAYPYDVPDYGSHHHHHH

SEQ ID NO: 46 - protein, 145 aa, Artificial Sequence
  Synthetic Peptide
QVQLQESGGGLVQAGGSLRLSCASSGFTFSGYQLAWFRQVAPGKEREFVAAIRWSGGSTWYADSVKGRFTISRDNAKNTVYLQMNSLKPEETAVYYCAASPLYSNSYYLPDNYDYWGQGTQVTVSSAAAYPYDVPDYGSHHHHHH

SEQ ID NO: 47 - protein, 145 aa, Artificial Sequence
  Synthetic Peptide
QVQLQESGGGLVQPGGSLRLSCAASGFTFDDYAIGWFRQAPGKEREGVSCISNSEGSTYYADSVKGRFTISRDNAENTMYLQMNSLKPEDTAVYYCAASPLFVAGICLSKYEYDYWGQGTQVTVSSAAAYPYDVPDYGSHHHHHH

SEQ ID NO: 48 - protein, 147 aa, Artificial Sequence
  Synthetic Peptide
QVQLQESGGGLVQAGSSLKLSCIASGDLFKSAGVGWFRQAPGKEREFVAAISWSGDSTYSADSVRGRFTISRDNAKNTVYLQMNSLKPEDTAVYYCAADGRNYGLGPLETDSYEYDYWGQGTQVTVSSAAAYPYDVPDYGSHHHHHH

SEQ ID NO: 49 - protein, 145 aa, Artificial Sequence
  Synthetic Peptide
QVQLQESGGGLVQAGGSLRISCAASGGIFSDYYMGWFRQAPGKEREFVAAISWSGRTTYYTNSVKGRFTISRDNAKNTLYLQMNNLKPEDTAVYRCAARPSNSGVYFSNTYDYNDWGQGTQVTVSSAAAYPYDVPDYGSHHHHHH

SEQ ID NO: 50 - protein, 145 aa, Artificial Sequence
  Synthetic Peptide
QVQLQESGGGLVQAGGSLRLSCAASGFTFDDYAIGWFRQAPGKEREGVSCISSSDGSTYYADSVKGRFTIARDNAKNTVYLQMNSLKPEDTAVYYCAASPLYYSGSYCRETEYDYWGQGTQVTVSSAAAYPYDVPDYGSHHHHHH

SEQ ID NO: 51 - protein, 139 aa, Artificial Sequence
  Synthetic Peptide
QVQLQESGGGLVQAGDSLRLSCAASGSYSIGWFRQAPGKEREFVAAISWSGGRTNYADSVKGRFTISRDNAKNTVYLQMNSLKPEDTAVYYCSAAIYDNVSSRIGSYTYWGQGTQVTVSSAAAYPYDVPDYGSHHHHHH

SEQ ID NO: 52 - protein, 140 aa, Artificial Sequence
  Synthetic Peptide
QVQLQESGGGLVQTGASLRLSCAASGSIFSIDTMGWYRQAPGKQRELVAAITRGGSTNYADSVKGRFTISRDNAKNTVYLQMNSLSPEDTAVYYCNANTGRLAVNYEHDYWGQGTQVTVSSAAAYPYDVPDYGSHHHHHH

SEQ ID NO: 53 - protein, 145 aa, Artificial Sequence
  Synthetic Peptide
QVQLQESGGGLVQAGGSLRISCAASGRTFSDYYMGWFRQAPGKEREFVAAISWSGRTTYYTNSVKGRFTISRDNAKNTLYLQMNNLKPEDTAVYRCAARPSNSGAYFARTHDYTDWGQGTQVTVSSAAAYPYDVPDYGSHHHHHH

SEQ ID NO: 54 - protein, 141 aa, Artificial Sequence
  Synthetic Peptide
QVQLQESGGGLVQPGGSLRLACAASGSVSSINDMGWYRQAPGKQREFVARISRGGLTNYKDSVKGRFTISRDNAKNQVYLQMNSLKPEDTAVYYCNADFRPSSGTFRLSDYWGQGTQVTVSSAAAYPYDVPDYGSHHHHHH

SEQ ID NO: 55 - DNA, 438 bp, Artificial Sequence
  Synthetic DNA
caggtgcagctgcaggagtctgggggaggattggtgcaggctgggggctctctgagactctcctgtgcaagctctggatttaccttcagtggctatcaattggcctggttccgccacgttgcaccagggaaggagcgtgagtttgtagccgctattaggtggagtggtggtatcacatggtatgcagactccgtgaagggccgattcaccatctctagagacaacgccaagaacacgatgtatctgcaaatgaacagcctgaaacctgaggaaacggccgtttattactgtgcagcaagtcccctctatagtaataattatctcctcccggataactatgactattggggccaggggacccaggtcaccgtctcctcagcggccgcatacccgtacgacgttccggactacggttcccaccaccatcaccatcactag

SEQ ID NO: 56 - DNA, 438 bp, Artificial Sequence
  Synthetic DNA
caggtgcagctgcaggagtctgggggaggcttggtgcagcctggggggtctctgagactctcctgtgcagccgctggattcactttcgattattatgccatagcctggttccgccaggccccagggaaggagcgcgagggggtctcatgtattagtagtagtgatggtaccacatactatgcagactccgtgaagggccgattcaccatctccaaggacaatgccaagaacacgatgtatctgcaaatgaacagcctgaaacctgaagacacggccgtttattactgtgcgacctcccccctctatagcaccaatgacaggtgtatttcggaagactatgactactggggccaggggacccaggtcaccgtctcctcagcggccgcatacccgtacgacgttccggactacggttcccaccaccatcaccatcactag

SEQ ID NO: 57 - DNA, 441 bp, Artificial Sequence
  Synthetic DNA
caggtgcagctgcaggagtctgggggaggcttggtgcagcctgggggatcagtgagactctcctgtgcagcctctggattcactttagatcgttttaccatcggctggttccgccaggtcccagggaaggagcgcgagggggtctcatgtattagtgaagttgctggtagcatatactatgcagactccgtgaagggccgattcaccatctccagagacaatgccaagaatacggtgtatctgcaaatgaacagcctgaaacctgaggacacggccgtttatacctgtgcagcagatccgttgctgtatcgcggggggggctgcctaatcccacatgaatatccctactggggccaggggacccaggtcaccgtctcctcagcggccgcatacccgtacgacgttccggactacggttcccaccaccatcaccatcactag

SEQ ID NO: 58 - DNA, 420 bp, Artificial Sequence
  Synthetic DNA
caggtgcagctgcaggagtctgggggaggcttggtgcagcctggggggtctctgagactctcctgtgcagcctctggattcaccttcagtaactatgccatgacctgggtccgccaggctccaggaaaggtcgtcgagtgggtctcacgtattcatagtggtgataatagcataacctacgcagagttcgtgaagggtcgattcaccatctccagagacaacgccaggaacacgctatatctgcaaatgaacagcctgaaacctgaggacacggccgtgtattactgcgccagagtagttcccgggttccaatatagatatgactactggggccaggggacccaggtcaccgtctcctcagcggccgcatacccgtacgacgttccggactacggttcccaccaccatcaccatcactag

SEQ ID NO: 59 - DNA, 441 bp, Artificial Sequence
  Synthetic DNA
caggtgcagctgcaggagtctgggggaggcttggtgcagcctggggggtctctaagactctcctgtgcagcctctggattcactttggattcgcataccataggctggttccgccaggccccagggaaggagcgcgagggggtctcaggtattaatagtagtgatggtagcacatactatgcagactccgtgaagggccgattcaccatctccagagacaatgccaagaacacggtgtatctgcaaatggacagcctgaaagttgaggacacggccgtttattactgtgcgacgacgcctttcagcctgcgtgggccgacctgggttacgccagatgagtttgactactggggccaggggacccaggtcaccgtctcctcagcggccgcatacccgtacgacgttccggactacggttcccaccaccatcaccatcactag

SEQ ID NO: 60 - DNA, 432 bp, Artificial Sequence
  Synthetic DNA
caggtgcagctgcaggagtctgggggaggcttggtgcaacctggggggtctctgagactctcctgtgaagcctctggacgcaccttcagtagctataccgtgggctggttccgccaggcttcagggaaagagcgtgagtttgtgggaactgttaattggagtggacaaatcacaacctatgcagactccgtgaagggccgattcaccatctccagacagtacgccgagaatacggtgtatctggaaatgaacagcctgaaaaatgaggacacggccgtttattactgtgcagcacatcaaggactgggaacgcctcgtacccccaaacagtatgattactggggccgcgggacccaggtcaccgtctcctcagcggccgcatacccgtacgacgttccggactacggttcccaccaccatcaccatcactag

SEQ ID NO: 61 - DNA, 444 bp, Artificial Sequence
  Synthetic DNA
caggtgcagctgcaggagtctgggggaggagtggtacaggatggaggctctctgagactctcctgtgcagcctctggacgcaccttcagtagctatgccatgggctggttccgccaggctccagggaaggagcgtgagtttgtagcagctattagctggagtggtgatagcacatactctgcagactccgtgaggggccgattcaccatctccagagacaacgccaagaacacggtgtatctgcaaatgaacagcctgaaacctgaggacacggccgtttattactgtgcagcagatggtaggaactacgggttggggccgctcgaaaccgactcatatgagtatgactactggggccaggggacccaggtcaccgtctcctcagcggccgcatacccgtacgacgttccggactacggttcccaccaccatcaccatcactag

SEQ ID NO: 62 - DNA, 441 bp, Artificial Sequence
  Synthetic DNA
caggtgcagctgcaggagtctgggggaggcttggtgcagcctggggggtctctgagactctcctgtgcagcctctggattgtctttgggttattatgccattggctggtttcgccaggccccagggaaggagcgcgaggtggtctcatgtgatagtgttgcggatggtagcacatactatgcagactcggtactgggccgattcaccatctccagagacaatcccaagaacacgatgtatttgcaaatgaacagcctgaaacctgaggacacggccgtttattactgtgcagcactcttcgactcagggttgggagactttccaccacctgttgaagactatgactattggggccaggggacccaggtcaccgtctcctcagcggccgcatacccgtacgacgttccggactacggttcccaccaccatcaccatcactag

SEQ ID NO: 63 - DNA, 438 bp, Artificial Sequence
  Synthetic DNA
caggtgcagctgcaggagtctgggggaggcttggtgcaggctggggggtctctgagactctcctgtgcagcctctggattcactttcgataattatgccataggctggttccgccaggccccagggaaggagcgtgagggggtctcatgtattggtagtagtgatggtagcacatactatgcagactccgtgaagggccgattcaccatctccagagacaacgccaagaacacgaagtatctgcaaatgaacagcctgaaacctgaggacacggccgtttattactgtgcagcatctccccttttcgtagctggtatatgcttgtcagaatatgattatgactactggggccaggggacccaggtcaccgtctcctcagcggccgcatacccgtacgacgttccggactacggttcccaccaccatcaccatcactag

SEQ ID NO: 64 - DNA, 432 bp, Artificial Sequence
  Synthetic DNA
caggtgcagctgcaggagtctgggggaggattggtgcaggctgggggctctctgagactctcctgtgcagcctctggacgcaccttcagtagcagtaccatgggctggttccgccaggctccagggaaagagcgtgagtttgtggcaactgttaattggaatggacaaatcacaacctatgcagactccgtgaagggccgattcaccatctccagacagtacgccgagaatacggtgtatctggacatgaacagcctgaaaaatgaggacacggccgtttattactgtgcagcacatcaagggctgggaacgcctcgtacccccaaacagtatgcttactggggccgcgggacccaggtcaccgtctcctcagcggccgcatacccgtacgacgttccggactacggttcccaccaccatcaccatcactag

SEQ ID NO: 65 - DNA, 444 bp, Artificial Sequence
  Synthetic DNA
caggtgcagctgcaggagtctgggggaggcgtggtgcagactggggggtctctgagactctcttgtgttgcctctggattcagtttcgaagactatggcataggctggtttcgccaggccccagggaaggaacgagagggagtctcatatattaaaaatacagatggtgatacaacctatgcagactccgtgaagggccgattcaccatctccagtgactacgccaagaatacggtgtatctgcagatgaacgacctggaacctcaggacgcggccgtttattactgtgcagcgagacccccacccctaaattactatagcccctatctcacgcccagagaatttagctactggggccaggggacccaggtcaccgtctcctcagcggccgcatacccgtacgacgttccggactacggttcccaccaccatcaccatcactag

SEQ ID NO: 66 - DNA, 402 bp, Artificial Sequence
  Synthetic DNA
caggtgcagctgcaggagtctgggggaggcttggtgcagcctggggggtctctgagactctcctgtgcagcctctggattcaccttcagtagctatgccatgagctgggtccgccaggctccaggaaaggggctagagtgggtcgcaactattggtggcggtggtgctgccagtaacgttgcagactccgtgaagggacgattcaccatctccagagacaacgccaagaacacgctgtatctgcaaatgaacagcctgaaacctgacgacacggccgtgtattactgtgaggaaagtcgacgagggacccggggccaggggacccaggtcaccgtctcctcagcggccgcatacccgtacgacgttccggactacggttcccaccaccatcaccatcactag

SEQ ID NO: 67 - DNA, 444 bp, Artificial Sequence
  Synthetic DNA
caggtgcagctgcaggagtctgggggaggattggtgcagactgggggctctctgagactctcctgtgcagcctctggacgcaccttcagtagctatgccatgggctggttccgccaggctccagggaaggagcgtgagtttgtagcagctattagctggagtggtgatagcacatactctgcagactccgtgaggggccgattcaccatctccagagacaacgccaagaacacggtgtatctgcaaatgaacagcctgaaacctgaggacacggccgtttattactgtgcagcagatggtaggaactacgggttggggccgctcgaaaccgactcatatgagtatgactactggggccaggggacccaggtcaccgtctcctcagcggccgcatacccgtacgacgttccggactacggttcccaccaccatcaccatcactag

SEQ ID NO: 68 - DNA, 429 bp, Artificial Sequence
  Synthetic DNA
caggtgcagctgcaggagtctgggggaggcttggtgcagcctggggggtctctgagactctcctgtgcagcctctggattcactttggacaattatgccataggctggttccgccaggccccagggaaggagcgcgagggggtctcatgtattagtagtagtgatggcagcgcatactatgcagactccgtgaagggccgattcaccatctccagagacaatgccaagaacacggtgaatctgcaaatgaacagcctgaaacctgaggacacggccgtttattactgtgcagcagatcctttctggggtaggtgccaggcatctgactatgcatactggggccaggggacccaggtcaccgtctcctcagcggccgcatacccgtacgacgttccggactacggttcccaccaccatcaccatcactag

SEQ ID NO: 69 - DNA, 441 bp, Artificial Sequence
  Synthetic DNA
caggtgcagctgcaggagtctgggggaggattggtgcaggctgggggctctctgagactctcctgtgcaccctctggacgcaccttcagtagcgatgccatgggctggttccgccaggttccagggaaggagcgtgagtttgtagcagctattagctggagtggtggtagcacatattatgcagactccgtgaagggccgattcaccatctccagagacaacgccaagaacacggtgtatctgcaaatggacagcctgaaacctgaggacacggccgtttattactgtgcagcatccctccttcgtagttactactcacccgatcatacctcatatgagtatgactactggggccaggggacccaggtcaccgtctcctcagcggccgcatacccgtacgacgttccggactacggttcccaccaccatcaccatcactag

SEQ ID NO: 70 - DNA, 444 bp, Artificial Sequence
  Synthetic DNA
caggtgcagctgcaggagtctgggggaggattggtgcaggctgggggctctctgagactctcctgtgtagcctctgaaagcaccttcagtccgtatggcatgggctggttccgccaggctccagggaaggagcgtgagtttgtagcagctattagctggagtggtgatagcacatactctgcagactccgtgaggggccgattcaccatctccagagacaacgccaagaacacggtgtatctgcaaatgaacagcctgaaacctgaggacacggccgtttattactgtgcagcagatggtaggaactacgggttggggccgctcgaaaccgactcatatgagtatgactactggggccaggggacccaggtcaccgtctcctcagcggccgcatacccgtacgacgttccggactacggttcccaccaccatcaccatcactag

SEQ ID NO: 71 - DNA, 432 bp, Artificial Sequence
  Synthetic DNA
caggtgcagctgcaggagtctgggggaggcttggtgcaggctggggactctctgagacttacctgtgcacaatctagacgcaccttcagtccctatgccatggcctggttccgccaggctccagggaaggagcgtgagtttgtagcagctgttacctggagtggtggtagcacatactatgcagactccgtgaagggccgattcaccgtctccagagacaacgccaagagcacgctgtatctgcaaatgaacagcctgaaacctgaggacacggccgtttattattgtgcagccagcctcttcgtaatagctggtacgcctaacgatgagtatgactactggggccaggggacccaggtcaccgtctcctcagcggccgcatacccgtacgacgttccggactacggttcccaccaccatcaccatcactag

SEQ ID NO: 72 - DNA, 438 bp, Artificial Sequence
  Synthetic DNA
caggtgcagctgcaggagtctggaggaggcttggtgcaggctggggggtctctgagactctcctgtgcagcctctggattcactttcgatgattatgccataggctggttccgccaggccccagggaaggagcgtgagggggtctcatgtattagtagtagtgatggtagcacatactatgcagactccgtgaagggccgattcaccatcgccagagacaacgccaagaacaccgtttatctgcaaatgaacagcctgaaacctgaggaaacggccgtttattactgtgcagcctcgccgctatactatagtggtagttactgccgtgagactgagtatgactactggggccaggggacccaggtcaccgtctcctcagcggccgcatacccgtacgacgttccggactacggttcccaccaccatcaccatcactag

SEQ ID NO: 73 - DNA, 441 bp, Artificial Sequence
  Synthetic DNA
caggtgcagctgcaggagtctgggggaggattggtgcaggctgggggctctctgagactctcctgtgcagcctctggacgcaccttcagtagtgatgccatgggctggttccgccaggctccagggaaggagcgtgagtttgtagcagctattaggtggagtggtggtagcacatactatgcagactccgtgaagggccgattcaccatctccagaggcaacgccaagaacacggtgtatctgcaaatgaacagcctgaaacctgaggacacggccgtttattactgtgcatcatccctccttaatagttactacccacccgatcatacctcatatgagtatgactactggggccaggggacccaggtcaccgtctcctcagcggccgcatacccgtacgacgttccggactacggttcccaccaccatcaccatcactag

SEQ ID NO: 74 - DNA, 444 bp, Artificial Sequence
  Synthetic DNA
caggtgcagctgcaggagtctgggggaggattggtgcaggctgggggctccctgagactctcctgtgcagcctctggacgctcctccagttcctacgccatcgcctggttccgccaggctccaggaaaggagcgtgaatttgcagcagctattagctggagtggtgatagcacatactctgcagactccgtgaggggccgattcaccatctccagagacaacgccaagaacacggtgtatctgcaaatgaacagcctgaaacctgaggacacggccgtttattactgtgcagcagatggtaggaactacgggttggggccgctcgaaaccgactcatatgagtatgactactggggccaggggacccaggtcaccgtctcctcagcggccgcatacccgtacgacgttccggactacggttcccaccaccatcaccatcactag

SEQ ID NO: 75 - DNA, 441 bp, Artificial Sequence
  Synthetic DNA
caggtgcagctgcaggagtctggaggaggattagtgcagcctgggggctctctgagactctcctgtgcagcctctggacgcactttcactaggtcgatcatgggctggttccaccaggctccagggaaggagcgtgagtttctagcaggcatttcgtggagtggggctaacacatactacgcagactccgtgaggggccggttcaccatctccagagacaacgccaagaacacggtgtctctgcaaatgaacagcctgaatcctgaggacacggccgtttattactgtgcagcagatccgtctcatcctgggtcgctaatttctactcgtcggtctgactatgactcctggggccgggggacccaggtcaccgtctcctcagcggccgcatacccgtacgacgttccggactacggttcccaccaccatcaccatcactag

SEQ ID NO: 76 - DNA, 447 bp, Artificial Sequence
  Synthetic DNA
caggtgcagctgcaggagtctgggggaggattggtgcaggctgggggctctctgagactctcctgtgcgccctctggacgcaccatcagtagcgtagatgtgggctggttccgccaggctccagggaaggagcgtgagtttgtagcacacgttacctggagtggtggtagcacatacgctgcagactccgtgaagggccgattcaccgtctccagagacaacgccaagaacacggtgtatctgcagatgaacagcctgaaacctgaggacacggccgtttattactgtgcagcggcattaaacggggcgttcagggatagctggtaccctgccttgtgggatgagtatgactactggggccaggggacccaggtcaccgtctcctcagcggccgcatacccgtacgacgttccggactacggttcccaccaccatcaccatcactag

SEQ ID NO: 77 - DNA, 432 bp, Artificial Sequence
  Synthetic DNA
caggtgcagctgcaggagtctgggggaggattggtgcaggctgggggctctctgagactctcctgtgaagcctctggacgcaccgccggtagcagtaccatagcctggttccgccaggctccagggaaagagcgtgagtttgtgacaactgttaattggagtggacaaatcacaacctatgcagactccgtgaagggccgattcaccatctccagacagtacgccgagaatacggtgtatctggaaatgaacagcctgaaaaatgaggacacggccgtttattactgtgcagcacatcaagggctgggaacgcctcgtacccccaaacagtatgattactggggccgcgggacccaggtcaccgtctcctcagcggccgcatacccgtacgacgttccggactacggttcccaccaccatcaccatcactag

SEQ ID NO: 78 - DNA, 444 bp, Artificial Sequence
  Synthetic DNA
caggtgcagctgcaggagtctgggggaggctcggtgcaggctggggactctctgagactctcctgtgcagcctctggacgcaccttcagtagttatgccatggcctggttccgccaggctccagggaaggaacgtgagtttgtagcaggaattcactggagtggtaccattacatactatgcagactccgtgaagggccgattcgccctctccagagacaacgccaagaacacggtctatctggaaatgaacagcctgaaacctgaggacacggccgtttattactgtgcagcaaataattaccactgttctacgtatgggtgtacgacgagtccagcgaggtatgactactggggccaggggacccaggtcaccgtctcctcagcggccgcatacccgtacgacgttccggactacggttcccaccaccatcaccatcactag

SEQ ID NO: 79 - DNA, 438 bp, Artificial Sequence
  Synthetic DNA
caggtgcagctgcaggagtctggaggaggcttggtgcagcctggggggtctctgagactctcctgtgcagcctctggattcactttggataattatgccataggctggttccgccaggccccagggaaggagcgcgagggggtctcatgtattagtagtagtgatggtagaacatactatgcagactccgtgaagggccgattcaccatctccagagacaatgccaagaacacgatgtatctgcaaatgaacagcctgaaacctgaggacacggccgtttattactgtgcgacctcccccctctatagcgactctgataggtgtatttgggaagagtatgactactggggccaggggacccaggtcaccgtctcctcagcggccgcatacccgtacgacgttccggactacggttcccaccaccatcaccatcactag

SEQ ID NO: 80 - DNA, 432 bp, Artificial Sequence
  Synthetic DNA
caggtgcagctgcaggagtctggaggaggattggtgcaggctgggggctctctgagactctcctgtgcagcctctggacgcaccttcggtagctatgccgtgggctggttccgccaggcttcagggaaagagcgtgagtttgtgggaactgttaattggagtggacaaatcacaacctatgcagactccgtgaagggccgattcaccatctccagacagtacgccgagaatacggtgtatctggaaatgaacagcctgaaaaatgaggacacggccgtttattactgtgcagcacatcaaggactgggaacgcctcgtacccccaaacagtatgattactggggccgcgggacccaggtcaccgtctcctcagcggccgcatacccgtacgacgttccggactacggttcccaccaccatcaccatcactag

SEQ ID NO: 81 - DNA, 444 bp, Artificial Sequence
  Synthetic DNA
caggtgcagctgcaggagtctgggggaggtctggtgcaaactgggggctctctgagactctcctgtacagcctctggacgcgcctttagtaggtatgccatgggctggttccgtcaggctccagggaaggagcgtgaatttgtagcagctattagctggagtggtgatagcacatactctgcagactccgtgaggggccgattcaccatctccagagacaacgccaagaacacggtgtatctgcaaatgaacagcctgaaacctgaggacacggccgtttattactgtgcagcagatggtaggaactacgggttggggccgctcgaaaccgactcatatgagtatgactactggggccaggggacccaggtcaccgtctcctcagcggccgcatacccgtacgacgttccggactacggttcccaccaccatcaccatcactag

SEQ ID NO: 82 - DNA, 420 bp, Artificial Sequence
  Synthetic DNA
caggtgcagctgcaggagtctgggggaggcttggtgcaggctggggactctctgagactctcctgtgcagcctctggaacgtattccatgggctggttccgccaggctccaggaaaggagcgtgagtttgtagcagctattagctggagcggtggacgcactaactatgcagactccgtgaagggccgattcaccatctccagagacaacgccaagaacacggtgtatctgcaaatgaacagcctgaaacctgaggacacggccgtttattactgttcagctgccatttacgataatgtctcatcgcgcattgggtcgtatacctactggggccaggggacccaggtcaccgtctcctcagcggccgcatacccgtacgacgttccggactacggttcccaccaccatcaccatcactag

SEQ ID NO: 83 - DNA, 435 bp, Artificial Sequence
  Synthetic DNA
caggtgcagctgcaggagtctgggggaggcttggtgcaggttggggggtctctgagactctcctgtgcagcctctggattcattttcgatgattatgccataggctgattccgccaggccccagggaaggagcgtgagggggtctcatgtattagtagtagtgatggtagcacatactatgcagactccgtgaagggccgattcaccatctccagagacaacgccaagaacacggtgtatctgcaaatgaacagcctgaaacctgaggacacggccgtttattactgtgcagcaggcgttatgagtagctggcgggattataaacactacggcatggactactggggcaaagggacccaggtcaccgtctcctcagcggccgcatacccgtacgacgttccggactacggttcccaccaccatcaccatcactag

SEQ ID NO: 84 - DNA, 435 bp, Artificial Sequence
  Synthetic DNA
caggtgcagctgcaggagtctggaggaggattcgtgcaggctgggggctctctgagactctcctgtgtagcctctggacgcaccttcagtacctatgacatgggctggttccgccaggctccagggaaggagcgtgaatttgtagcaagtattcttataggtggcggcacatactatgcagactccgtgaagggccgattcaccatctccagagacaacgccaagaacacggtgtatctacaaatgaacagcctgaaacctgaggacacggccgtttattactgtgcatatcgtgccccttcgagtggtgcttactactccaaccaacgtgcctatgactactggggccaggggacccaggtcaccgtctcctcagcggccgcatacccgtacgacgttccggactacggttcccaccaccatcaccatcactag

SEQ ID NO: 85 - DNA, 444 bp, Artificial Sequence
  Synthetic DNA
caggtgcagctgcaggagtctgggggaggattggtgcaggctggggactctctgagactctcctgtgcagcctctggacgcaccttcagtaactatgccatgggctggttccgccaggagccagggaaggagcgtgagtttgtagcaggtattaactggagtggtgatagcacatactatgcagactccgtgaagggccgattcaccatctccagagacaacgccaagaatacgctgtatctgcaaatgaacagcctgaaacctgaggacacggccgtttattactgtgcagcaggtttattccatggtagctggtccgagagaccggaagccataagtgagtatgactactggggccaggggacccaggtcaccgtctcctcagcggccgcatacccgtacgacgttccggactacggttcccaccaccatcaccatcactag

SEQ ID NO: 86 - DNA, 441 bp, Artificial Sequence
  Synthetic DNA
caggtgcagctgcaggagtctgggggaggattgacgcaggctgggggctctctgagactctcctgtgcagcctctggacgcactttcactaggtcgatcatgggctggttccgccaggctccagggaaggagcgtgagtttctagcagccattacgtggagtgggggtaacacatactacgcagactccgtgaggggccggttcaccatctccagagacaacgccaagaacacggtgtctctgcaaatgaacagcctgaaacctgaggacacggccgtttattattgtgcagcagatccgtctcatcttgggtcgctaatttctactcgtcggtctgactatgactcctggggccgggggacccaggtcaccgtctcctcagcggccgcatacccgtacgacgttccggactacggttcccaccaccatcaccatcactag

SEQ ID NO: 87 - DNA, 447 bp, Artificial Sequence
  Synthetic DNA
caggtgcagctgcaggagtctgggggaggattggtgcaggctggggactctctgagactctcctgtgcagcctctggacgcaccttcagtagctatgtgatgggctggttccgccaggctccggggaaggagcgtgagtttgtagcagctattagctggagtggtggtagcacatactatgcagactccgtgaagggccgattcaccatctccagagacaacgccaagaacacggtgtatctgcaaatgaacagcctgaaacctgaggacacggccgtttattactgtgcagccgcagatccaccctctttctatagcgactatgactggccccgagaccatgagtatgactactggggccaggggacccaggtcaccgtctcctcagcggccgcatacccgtacgacgttccggactacggttcccaccaccatcaccatcactag

SEQ ID NO: 88 - DNA, 441 bp, Artificial Sequence
  Synthetic DNA
caggtgcagctgcaggagtctggaggaggattggtgcaagctgggggctctctgagactctcctgtgcaccctctggacgcaccttcagtagcgatgccatgggctggttccgccaggttccagggaaggagcgtgagtttgtagcagctattagctggagtggtggtagcacatattatgcagactccgtgaagggccgattcaccatctccagagacaacgccaagaacacggtgtatctgcaaatggacagcctgaaacctgaggacacggccgtttattactgtgcagcatccctccttcgtagttactacccacccgatcatacctcgtatgagtatgactactggggccaggggacccaggtcaccgtctcctcagcggccgcatacccgtacgacgttccggactacggttcccaccaccatcaccatcactag

SEQ ID NO: 89 - DNA, 447 bp, Artificial Sequence
  Synthetic DNA
caggtgcagctgcaggagtctggaggaggcttggtacaagctggggggtctctgagactctcctgtgctgcctctggagtcactttcgatagcgctgccatggcctggttccgccaagttccagggaaggagcgtgagtttgttgctgtgattagtaccgagagtggtgggaggacagaccacgcagactccgtgaagggccgattcctcatctccagagataacgcccgtcatatggtgtatctgcaaatgaatagcctgaaccctgaggacacggccgtttattactgtgcgtcaagtcttctctactgctcagcctctgggtgttacgcgaaccgtgactcgtatgactactggggccaggggacccaggtcaccgtctcctcagcggccgcatacccgtacgacgttccggactacggttcccaccaccatcaccatcactag

SEQ ID NO: 90 - DNA, 432 bp, Artificial Sequence
  Synthetic DNA
caggtgcagctgcaggagtctggaggaggattggtgcaggctgggggctctctgagactctcctgtgaagcctctggacgcaccttcagtagcagtaccatgggctggttccgccaggctccagggaaagagcgtgagtttgtggcaactgttaattggaatggacaaatcacaacctatgcagactccgtgaagggccgattcaccatctccagacagtacgccgagaatacggtgtatctggacatgaacagcctgaaaaatgaggacacggccgtttattactgtgcagcacatcaagggctgggaacgcctcgtacccccaaacagtatgcttactggggccgcgggacccaggtcaccgtctcctcagcggccgcatacccgtacgacgttccggactacggttcccaccaccatcaccatcactag

SEQ ID NO: 91 - DNA, 438 bp, Artificial Sequence
  Synthetic DNA
caggtgcagctgcaggagtctgggggaggcttggtgcaggctggggggtctctgagactctcctgtgcagcctctggattcactttcgatgattatgccataggctggttccgccaggccccaggaagagagcgtgagggggtctcatgtattagtgacagtgatggtagcacatactatgcagactccgtgaagggccgattcaccatctccagagacaacgccaagaacacaatgtatctgcaaatgaacagcctgaaacctgaggacacggccgtttattactgtgcagcatctccccttttcgtagctggtatatgcttgtcaaaatatgagtatgactactggggccaggggacccaggtcaccgtctcctcagcggccgcatacccgtacgacgttccggactacggttcccaccaccatcaccatcactag

SEQ ID NO: 92 - DNA, 435 bp, Artificial Sequence
  Synthetic DNA
caggtgcagctgcaggagtctggaggaggattggtacagactggggactctctgagactctcctgtgcaacctctggcggcaccttcagtggttatgaactggcctggttccgccaggctccagggaaggagcgtgagttcgtagcacatattacccggagtggtcgtgctacatactatttagactccgtgaagggccgattcaccatctccagagacaacgccaggaacacggtgtatctgcaaatgaacagcctgaaacctgaggacacggccgtttattactgtgcagcgtccctcttaataaatccggtgggggttggacaaaatgagtatgactactggggccaggggacccaggtcaccgtctcctcagcggccgcatacccgtacgacgttccggactacggttcccaccaccatcaccatcactag

SEQ ID NO: 93 - DNA, 432 bp, Artificial Sequence
  Synthetic DNA
caggtgcagctgcaggagtctggaggaggattggtgcaggctgggggctctttgagactctcctgtgcagcctctggacttaccttcggtaactgtgacttggcctggttccgccaggctccagggaaggagcgtgaaactgtagcagctgttaggcggagtgatggtgctacatactatgcagactccgtgaagggccgattcaccatctccagagacaacgccaagaacacggtgtatctccaaatgaacagcctgaaacctgaggacacggccgtttattactgtgcagcatccttacttacgtcggcgactatcatccaacctgagtatgactactggggccaggggacccaggtcaccgtctcctcagcggccgcatacccgtacgacgttccggactacggttcccaccaccatcaccatcactag

SEQ ID NO: 94 - DNA, 444 bp, Artificial Sequence
  Synthetic DNA
caggtgcagctgcaggagtctgggggaggattggtgcaggctgggggatctctgagactctcctgtgcagcctctggacgcaccttcagtacgtattccatgggctggttccgccaggctccagggaaggagcgtgagtttgtagcagctattagctggagtggtgatagcacatactctgcagactccgtgaggggccgattcaccatctccagagacaacgccaagaacacggtgtatctgcaaatgaacagcctgaaacctgaggacacggccgtttattactgtgcagcagatggtaggaactacgggttggggccgctcgaaaccgactcatatgagtatgactactggggccaggggacccaggtcaccgtctcctcagcggccgcatacccgtacgacgttccggactacggttcccaccaccatcaccatcactag

SEQ ID NO: 95 - DNA, 423 bp, Artificial Sequence
  Synthetic DNA
caggtgcagctgcaggagtctgggggaggcttggtgtagcctggggggtctctaagactctcctgcgcagcctctacaagcatcttcagtttcgatttcatggcctggtaccgccaggctccaggggagcagcgcgacttggtcgcagcacttactagtaatggtgccacaaactatgcagactccgtgaagggccgattcaccatctccagagacaacgcctagaacaacgtgtatctgcaaatggacagcctgaaacctgaggacacggccgtctattactgcaatgcagctctacgaactgattcctgggacaggcgggaagactactggggccaggggacccaggtcaccgtctcctcagcggccgcatacccgtacgacgttccggactacggttcccaccaccatcaccatcactag

SEQ ID NO: 96 - DNA, 438 bp, Artificial Sequence
  Synthetic DNA
caggtgcagctgcaggagtctgggggaggcttggtgcagcctggggggtctctgagactctcctgtgcagcctctggattcactttggattattatgccataggctggttccgccaggccccagggaaggagcgcgagggggtctcatgtattagtagtaatgatggtagcacatactatgcagactccgtgaagggccgattcaccatctccagagacaatgccaagaacacggtgtatctgcaaatgaacagcctgaaacctgaggacacggccgtttattactgtgcgacgtccccactatactatagtggtagttactgtcgtgccgacgagtatgactactggggccaggggacccagatcaccgtctcctcagcggccgcatacccgtacgacgttccggactacggttcccaccaccatcaccatcactag

SEQ ID NO: 97 - DNA, 432 bp, Artificial Sequence
  Synthetic DNA
caggtgcagctgcaggagtctgggggaggattggtgcaggctggggactctctgagactctcctgtgaagcctctggacgcaccttcagtagctataccgtgggctggttccgccaggcttcagggaaagagcgtgagtttgtgggaactgttaattggagtggacaaatcacaacctatgcagactccgtgaagggccgattcaccatctccagacagtacgccgagaatacggtgtatctggaaatgaacagcctgaaaaatgaggacacggccgtttattactgtgcagcacatcaaggactgggaacgcctcgtacccccaaacagtatgattactggggccgcgggacccaggtcaccgtctcctcagcggccgcatacccgtacgacgttccggactacggttcccaccaccatcaccatcactag

SEQ ID NO: 98 - DNA, 435 bp, Artificial Sequence
  Synthetic DNA
caggtgcagctgcaggagtctgggggaggcttggtgcagcctgggggcgctctgagactctcctgtgcagcctctggattcactttggattattatgccataggctggttccgccaggccccagggaaggagcgcgaggcagtctcatgtattagtagtagtgatggtagcacatactatgcagactccgtgaagggccgattcaccatctccagagacaatgccaagaacacggtgtatctgcaaatgaacagcctgaaacctgaggacacggccgtttattactgtgcgacagacccgttctatagtgatattaactgcaagtcatatgagtatgcctattggggccaggggacccaggtcaccgtctcctcagcggccgcatacccgtacgacgttccggactacggttcccaccaccatcaccatcactag

SEQ ID NO: 99 - DNA, 435 bp, Artificial Sequence
  Synthetic DNA
caggtgcagctgcaggagtctgggggaggattggtacagactggggactctctgagactctcctgtgcaacctctggcggcaccttcagtagttatgagatggcctggttccgccaggctccagggaaggagcgtgagttcgtagcacatattacccggagtggtcgtagtgcatactatttagactccgtgaagggccgattcaccatctccagagacaacgccaggaacacggtgtatctgcaaatgaacagcctgaaacctgaggacacggccgtttattactgtgcagcgtccctcttaataaatccggtgggggttggacaaaatgagtatgactactggggccaggggacccaggtcaccgtctcctcagcggccgcatacccgtacgacgttccggactacggttcccaccaccatcaccatcactag

SEQ ID NO: 100 - DNA, 438 bp, Artificial Sequence
  Synthetic DNA
caggtgcagctgcaggagtctgggggaggattggtgcaggctgggggctctctgagactctcctgtgcaagctctggatttaccttcagtggctatcaattggcctggttccgccaggttgcaccaggcaaggagcgtgagtttgtagccgctattaggtggagtggtggtagcacatggtatgcagactccgtgaagggccgattcaccatctctagagacaacgccaagaacacggtgtatctgcaaatgaacagcctgaaacctgaggaaacggccgtttattactgtgcagcaagtcccttgtatagtaatagttactacctcccggataactatgactattggggccaggggacccaggtcaccgtctcctcagcggccgcatacccgtacgacgttccggactacggttcccaccaccatcaccatcactag

SEQ ID NO: 101 - DNA, 438 bp, Artificial Sequence
  Synthetic DNA
caggtgcagctgcaggagtctgggggaggcttggtgcagcctggggggtctctgagactctcctgtgcagcctctggattcactttcgatgattatgccataggctggttccgccaggccccagggaaggagcgtgagggggtctcatgtattagtaatagtgagggtagcacatactatgcagactccgtgaagggccgattcaccatctccagagacaacgccgagaacacgatgtatctgcaaatgaacagcctgaaacctgaggacacggccgtttattactgtgcagcatctccccttttcgtagctggtatatgcttgtcaaaatatgagtatgactactggggccaggggacccaggtcaccgtctcctcagcggccgcatacccgtacgacgttccggactacggttcccaccaccatcaccatcactag

SEQ ID NO: 102 - DNA, 444 bp, Artificial Sequence
  Synthetic DNA
caggtgcagctgcaggagtctgggggaggattggtgcaggctgggagttctctgaaactctcctgtatagcctccggagatctcttcaaaagcgccggagtgggttggttccgccaggctccagggaaggagcgtgagtttgtagcagctattagctggagtggtgatagcacatactctgcagactccgtgaggggccgattcaccatctccagagacaacgccaagaacacggtgtatctgcaaatgaacagcctgaaacctgaggacacggccgtttattactgtgcagcagatggtaggaactacgggttggggccgctcgaaaccgactcatatgagtatgactactggggccaggggacccaggtcaccgtctcctcagcggccgcatacccgtacgacgttccggactacggttcccaccaccatcaccatcactag

SEQ ID NO: 103 - DNA, 438 bp, Artificial Sequence
  Synthetic DNA
caggtgcagctgcaggagtctgggggaggattggtgcaggctgggggctctctgagaatttcctgtgcagcctctggaggcatcttcagtgactattacatgggctggttccgccaggctccagggaaggagcgtgagtttgtggcagctattagctggagtggtagaaccacatactatacaaactccgtgaagggccgattcaccatctccagagacaacgccaagaacacgctgtatctgcagatgaacaacctgaaacctgaggacacggccgtttatcgctgcgcagcccgaccctctaatagcggtgtttacttctctaacacatatgactataacgactggggccaggggacccaggtcaccgtctcctcagcggccgcatacccgtacgacgttccggactacggttcccaccaccatcaccatcactag

SEQ ID NO: 104 - DNA, 438 bp, Artificial Sequence
  Synthetic DNA
caggtgcagctgcaggagtctgggggaggcttggtgcaggctggggggtctctgagactctcctgtgcagcctctggattcactttcgatgattatgccataggctggttccgccaggccccagggaaggagcgtgagggggtctcatgtattagtagtagtgatggtagcacatactatgcagactccgtgaagggccgattcaccatcgccagagacaacgccaagaacaccgtttatctgcaaatgaacagcctgaaacctgaggacacggccgtttattactgtgcagcctcgccgctatactatagtggtagttactgccgtgagactgagtatgactactggggccaggggacccaggtcaccgtctcctcagcggccgcatacccgtacgacgttccggactacggttcccaccaccatcaccatcactag

SEQ ID NO: 105 - DNA, 420 bp, Artificial Sequence
  Synthetic DNA
caggtgcagctgcaggagtctgggggaggattggtgcaggctggggactctctgagactctcctgtgcagcctctggaagctattccatcgggtggttccgccaggctccaggaaaggagcgtgagtttgtagcagctattagctggagcggtggacgcactaactatgcagactccgtgaagggccgattcaccatctccagagacaacgccaagaacacggtgtatctgcaaatgaacagcctgaaacctgaggacacggccgtttattactgttcagctgccatttacgataatgtctcatcgcgcattgggtcgtatacctactggggccaggggacccaggtcaccgtctcctcagcggccgcatacccgtacgacgttccggactacggttcccaccaccatcaccatcactag

SEQ ID NO: 106 - DNA, 423 bp, Artificial Sequence
  Synthetic DNA
caggtgcagctgcaggagtctggaggaggattggtgcagactggggcctctctgagactctcctgtgcagcctctggaagcatcttcagtatcgataccatgggctggtaccgccaggctccagggaagcagcgcgagttggtcgcagctattactaggggtggtagcacaaattatgcagactccgtgaagggccgattcaccatctccagagacaacgccaagaacacggtgtatctgcaaatgaacagcctgagtcctgaggacacggccgtctattactgtaatgccaatacgggaaggctcgcggtaaattatgaacatgactactggggccaggggacccaggtcaccgtctcctcagcggccgcatacccgtacgacgttccggactacggttcccaccaccatcaccatcactag

SEQ ID NO: 107 - DNA, 438 bp, Artificial Sequence
  Synthetic DNA
caggtgcagctgcaggagtctgggggaggattggtgcaggctgggggctctctgagaatttcctgcgcagcctctggacgcaccttcagtgactattacatgggctggttccgccaggctccaggaaaggagcgtgagtttgtggcagctattagctggagtggtagaaccacatactatacaaactccgtgaagggccgattcaccatctccagagacaacgccaagaacacgctgtatctgcaaatgaacaacctgaaacctgaggacacggccgtttatcgctgcgcagcccgaccctctaatagcggtgcttacttcgctaggacccatgactataccgactggggccaggggacccaggtcaccgtctcctcagcggccgcatacccgtacgacgttccggactacggttcccaccaccatcaccatcactag

SEQ ID NO: 108 - DNA, 426 bp, Artificial Sequence
  Synthetic DNA
caggtgcagctgcaggagtctgggggaggcttggtgcagcctggggggtctctgagactcgcctgtgcagcctctggaagcgtcagtagtatcaatgacatgggctggtaccgccaggctccagggaagcagcgcgagttcgtcgcacggattagtcgtggtggtcttacaaactataaagactccgtgaagggccgattcaccatctccagagacaacgccaagaaccaggtgtatctgcaaatgaacagcctgaaacctgaggacacggccgtctattactgtaatgcagattttcgcccttctagtggtacgttccgactcagtgattactggggccaggggacccaggtcaccgtctcctcagcggccgcatacccgtacgacgttccggactacggttcccaccaccatcaccatcactag